(19) United States
(12) Patent Application Publication
(10) Pub. No.: US 2025/0289860 A1
(43) Pub. Date: Sep. 18, 2025

(54) POLYPEPTIDES AND USES THEREOF

(71) Applicant: MEDIMMUNE LIMITED, Cambridge (GB)

(72) Inventors: Maria Aleksandra BEDNAREK, Cambridge (GB); Sivaneswary GENAPATHY, Cambridge (GB)

(21) Appl. No.: 19/052,168

(22) Filed: Feb. 12, 2025

Related U.S. Application Data
(63) Continuation of application No. 17/551,701, filed on Dec. 15, 2021, now Pat. No. 12,371,464.
(60) Provisional application No. 63/125,996, filed on Dec. 16, 2020.

(51) Int. Cl. C07K 14/575; A61K 38/00; A61P 3/00; A61P 3/04; A61P 3/10

(52) U.S. Cl. CPC C07K 14/575; A61P 3/00; A61P 3/04; A61P 3/10; A61K 38/00; C07K 2319/31

(57) ABSTRACT

Disclosed are polypeptides which are pramlintide analogues and uses thereof. In particular, the present invention relates to polypeptides of SEQ ID NO 2 which are pramlintide analogues conjugated to half-life extending moieties such as albumin binding moieties and uses thereof.

### SEQUENCE LISTING

The content of the electronically submitted sequence listing (Name: 2943_2840004; Size: 1,137,670 bytes; and Date of Creation: Apr. 28, 2025) filed with the application is incorporated herein by reference in its entirety.

### FIELD OF THE INVENTION

The present invention relates to polypeptides which are pramlintide analogues and uses thereof. In particular, the present invention relates to polypeptides which are pramlintide analogues conjugated to half-life extending moieties such as albumin binding moieties and uses thereof

### BACKGROUND

Pramlintide is a synthetic analogue of human amylin with three proline substitutions, at positions 25, 28 and 29. As a result of these substitutions, pramlintide has a reduced propensity to form amyloid fibrils, thereby overcoming a physicochemical liability of native human amylin (Kruger D F, Gloster M A. Pramlintide for the treatment of insulin-requiring diabetes mellitus: rationale and review of clinical data. Drugs. 2004; 64 (13): 1419-32).

Pramlintide is clinically used in amylin replacement therapies and simulates the important glucoregulatory actions of amylin. These glucoregulatory actions complement those of insulin by regulating the rate of appearance of glucose in the circulation, and are achieved through three primary mechanisms: slowing the rate of gastric emptying, suppression of post-meal glucagon secretion and suppression of food intake (Roth J D et. al. GLP-1R and amylin agonism in metabolic disease: complementary mechanisms and future opportunities. Br J Pharmacol. 2012; 166 (1): 121-136). Pramlintide has been used as an adjunct to insulin in patients with diabetes who have failed to reach desired glucose control despite optimal insulin therapy (Pullman J, et. al. Pramlintide is used in the management of insulin-using patients with type 2 and type 1 diabetes. Vasc Health Risk Manag. 2006; 2 (3): 203-212).

Pharmacokinetic studies show that the terminal half-life of amylin in rats is around 13 minutes, and the half-life for pramlintide in human is ˜20-45 minutes (Roth J D et. al. GLP-1R and amylin agonism in metabolic disease: complementary mechanisms and future opportunities. Br J Pharmacol. 2012; 166 (1): 121-136).

There remains a need for pramlintide analogues which retain amylin agonist activity and provide advantages such as extended half-life and reduced fibrillation tendency.

### SUMMARY OF INVENTION

The present invention relates to polypeptides that are pramlintide analogues conjugated to albumin binding moieties (e.g. lipids).

Thus, in one aspect, there is provided a polypeptide, or a pharmaceutically acceptable salt thereof, comprising the amino acid sequence:
Xaa (−4)-Xaa (−3)-Xaa (−2)-Xaa (−1)-Xaa 1-Cys 2-Asn 3-Xaa 4-Ala 5-Thr 6-Cys 7-Ala 8-Thr 9-Gln 10-Arg 11-Leu 12-Ala 13-Xaa 14-Xaa 15 -Xaa 16-Xaa 17-His 18-Ser 19-Xaa 20-Xaa 21-Xaa 22-Xaa 23-Xaa 24 -Xaa 25-Xaa 26-Xaa 27-Xaa 28-Xaa 29-Thr 30-Xaa 31-Xaa 32-Xaa 33 -Xaa 34-Xaa 35-Xaa 36-Xaa 37-amide [SEQ ID NO:2], wherein:
Xaa (−4) is Lys (albumin binding moiety) or is absent;
Xaa (−3) is Gly or is absent;
Xaa (−2) is Gly or is absent;
Xaa (−1) is Gly, (albumin binding moiety), Lys (albumin binding moiety) or is absent;
Xaa 1 is Lys, Lys (albumin binding moiety), (albumin binding moiety) or is absent;
Xaa 4 is Thr, Ile or Ala;
Xaa 14 is Asn, His, Glu, 2,4-diaminobutanoic acid (Dab), or an alpha methyl amino acid;
Xaa 15 is Phe or Trp;
Xaa 16 is Leu or D-Leu (dL);
Xaa 17 is Val, Ser, Glu, Arg, (2S,4R)-4-hydroxypyrrolidine-2-carboxylic acid (Hyp), Dab or an alpha methyl amino acid (e.g. 2-amino-2-methylpropanoic acid [Aib]);
Xaa 20 is Ser, Ile, Pro or an alpha methyl amino acid (e.g. (S)-2-amino-3-hydroxy-2-methylpropanoic acid [aMeSer]);
Xaa 21 is Asn, Dab, His, Pro, Ser, Arg, Lys, Gly, Glu, Ala, Hyp or an alpha methyl amino acid (e.g. Aib);
Xaa 22 is Asn, His, Hyp, Dab or an alpha methyl amino acid (e.g. Aib);
Xaa 23 is Phe, Hyp or an alpha methyl amino acid (e.g. (S)-2-amino-2-methyl-3-phenylpropanoic acid [aMePhe]);
Xaa 24 is Gly, Pro, Hyp or an alpha methyl amino acid (e.g. Aib);
Xaa 25 is Pro, Ala, Hyp or an alpha methyl amino acid (e.g. Aib);
Xaa 26 is Ile, D-Ile (dl), Arg, Hyp or an alpha methyl amino acid (e.g. Aib);
Xaa 27 is Leu, dL, Hyp or an alpha methyl amino acid (e.g. Aib);
Xaa 28 is Pro, D-Pro (dP), Ser, Hyp or an alpha methyl amino acid (e.g. Aib);
Xaa 29 is Pro, Hyp or an alpha methyl amino acid (e.g. Aib);
Xaa 31 is Asn, Glu, His, Arg, Pro, Dab or an alpha methyl amino acid (e.g. Aib);
Xaa 32 is Val, Hyp, Dab or an alpha methyl amino acid (e.g. Aib);
Xaa 33 is Gly, Pro, Hyp or an alpha methyl amino acid (e.g. Aib);
Xaa 34 is Ser, Pro, His, Hyp or an alpha methyl amino acid (e.g. Aib);
Xaa 35 is Asn, Pro, Arg, Glu, Dab, Hyp or an alpha methyl amino acid (e.g. Aib);
Xaa 36 is Thr, Hyp or an alpha methyl amino acid (e.g. Aib); and
Xaa 37 is Tyr, Pro, Hyp or an alpha methyl amino acid (e.g. Aib),
   and wherein the polypeptide comprises at least one albumin binding moiety.

In another aspect, there is provided a lipidated polypeptide, or a pharmaceutically acceptable salt thereof, comprising the amino acid sequence:
Xaa (−4)-Xaa (−3)-Xaa (−2)-Xaa (−1)-Xaa 1-Cys 2-Asn 3-Xaa 4-Ala 5-Thr 6-Cys 7-Ala 8-Thr 9-Gln 10-Arg 11-Leu 12-Ala 13-Xaa 14-Xaa 15-Xaa 16-Xaa 17-His 18-Ser 19-Xaa 20-Xaa 21-Xaa 22-Xaa 23-Xaa 24-Xaa 25-Xaa 26-Xaa 27-Xaa 28-Xaa 29-Thr 30-Xaa 31-Xaa 32-Xaa 33-Xaa 34-Xaa 35-Xaa 36-Xaa 37-amide [SEQ ID NO: 159], wherein:
   Xaa (−4) is Lys (linker-lipid) or is absent;
   Xaa (−3) is Gly or is absent;
   Xaa (−2) is Gly or is absent;
   Xaa (−1) is Gly, (linker-lipid), Lys (linker-lipid) or is absent;
   Xaa 1 is Lys, Lys (linker-lipid), (linker-lipid) or is absent;
   Xaa 4 is Thr, Ile or Ala;
   Xaa 14 is Asn, His, Glu, 2,4-diaminobutanoic acid (Dab), or an alpha methyl amino acid;
   Xaa 15 is Phe or Trp;
   Xaa 16 is Leu or D-Leu (dL);
   Xaa 17 is Val, Ser, Glu, Arg, (2S,4R)-4-hydroxypyrrolidine-2-carboxylic acid (Hyp), Dab or an alpha methyl amino acid (e.g. 2-amino-2-methylpropanoic acid [Aib]);
   Xaa 20 is Ser, Ile, Pro or an alpha methyl amino acid (e.g. (S)-2-amino-3-hydroxy-2-methylpropanoic acid [aMeSer]);
   Xaa 21 is Asn, Dab, His, Pro, Ser, Arg, Lys, Gly, Glu, Ala, Hyp or an alpha methyl amino acid (e.g. Aib);
   Xaa 22 is Asn, His, Hyp, Dab or an alpha methyl amino acid (e.g. Aib);
   Xaa 23 is Phe, Hyp or an alpha methyl amino acid (e.g. (S)-2-amino-2-methyl-3-phenylpropanoic acid [aMePhe]);
   Xaa 24 is Gly, Pro, Hyp or an alpha methyl amino acid (e.g. Aib);
   Xaa 25 is Pro, Ala, Hyp or an alpha methyl amino acid (e.g. Aib);
   Xaa 26 is Ile, D-Ile (dl), Arg, Hyp or an alpha methyl amino acid (e.g. Aib);
   Xaa 27 is Leu, dL, Hyp or an alpha methyl amino acid (e.g. Aib);
   Xaa 28 is Pro, D-Pro (dP), Ser, Hyp or an alpha methyl amino acid (e.g. Aib);
   Xaa 29 is Pro, Hyp or an alpha methyl amino acid (e.g. Aib);
   Xaa 31 is Asn, Glu, His, Arg, Pro, Dab or an alpha methyl amino acid (e.g. Aib);
   Xaa 32 is Val, Hyp, Dab or an alpha methyl amino acid (e.g. Aib);
   Xaa 33 is Gly, Pro, Hyp or an alpha methyl amino acid (e.g. Aib);
   Xaa 34 is Ser, Pro, His, Hyp or an alpha methyl amino acid (e.g. Aib);
   Xaa 35 is Asn, Pro, Arg, Glu, Dab, Hyp or an alpha methyl amino acid (e.g. Aib);
   Xaa 36 is Thr, Hyp or an alpha methyl amino acid (e.g. Aib);
   Xaa 37 is Tyr, Pro, Hyp or an alpha methyl amino acid (e.g. Aib).

In yet another aspect, there is provided a polypeptide as set forth in Table 4.

In yet another aspect, there is provided a pharmaceutical composition comprising a polypeptide, a lipidated polypeptide or pharmaceutically acceptable salt of the invention and a pharmaceutically acceptable excipient.

In another aspect, there is provided a method for treating a disease or disorder in a subject comprising administering a polypeptide, a lipidated polypeptide, pharmaceutically acceptable salt or a pharmaceutical composition of the invention.

In a further aspect, there is provided a method for the production of a polypeptide or a lipidated polypeptide described herein.

In a further aspect, there is provided an article of manufacture comprising a polypeptide, a lipidated polypeptide, a pharmaceutically acceptable salt or a pharmaceutical composition of the invention.

In a further aspect, there is provided a kit comprising a polypeptide, a lipidated polypeptide, a pharmaceutically acceptable salt or a pharmaceutical composition of the invention, optionally further comprising instructions for use.

Aspects and embodiments of the invention are set out in the appended claims. These and other aspects and embodiments of the invention are also described herein.

### Brief Description of Sequence Listing

TABLE 1
Compound Sequence Listing

| SEQ | |
|---|---|
| ID NO. | Full sequence |
| 1 | K[CNTATC]ATQRLANFLVHSSNNFGPIL |
| (Pramlintide) | PPTNVGSNTY-amide |
| 3 | C18diacid-γE-K[CNTATC]ATQRLAN |
| | FLVHSSNNFGPILPPTNVGSNTY-amide |
| 4 | C18diacid-γE-γE-GGG-K[CNTATC] |
| | ATQRLANFLVHSSNNFGPILPPTNVGSNT |
| | Y-amide |
| 5 | K(γE-γE-C18diacid)[CNTATC]ATQ |
| | RLANFLVHSSNNFGPILPPTNVGSNTY- |
| | amide |
| 6 | K(O2Oc-O2Oc-γE-C18diacid) |
| | [CNTATC]ATQRLANFLVHSSNNFGPILP |
| | PTNVGSNTY-amide |
| 7 | K(O2Oc-O2Oc-γE-C18diacid)GGGK |
| | [CNTATC]ATQRLANFLVHSSNNFGPILP |
| | PTNVGSNTY-amide |
| 8 | K(γE-γE-C18diacid)[CNTATC]ATQ |
| | RLANFLVHSS(Dab)NFPAILSPTNVGSN |
| | TY-amide |
| 9 | K(γE-γE-C18diacid)[CNTATC]ATQ |
| | RLANFLVHSS(Aib)NFPAILSPTNVGSN |
| | TY-amide |
| 10 | C18diacid-γE-[CNTATC]ATQRLAEF |
| | LRHSSNN(αMePhe)GPILPPTNVGSNT |
| | Y-amide |
| 11 | K(γE-γE-C18diacid)[CNTATC]ATQ |
| | RLAEFLRHSSNN(αMePhe)GPILPPTNV |
| | GSNTY-amide |
| 12 | K(γE-C18diacid)K[CNTATC]ATQRL |
| | AEFLRHSSNN(αMePhe)GPILPPTNVGS |
| | NTY-amide |
| 13 | K(γE-γE-C18diacid)[CNTATC]ATQ |
| | RLANFLVHSSNN(αMePhe)GPILPPTNV |
| | GSNTY-amide |
| 14 | K(γE-C18diacid)K[CNTATC]ATQRL |
| | ANFLVHSSNN(αMePhe)GPILPPTNVGS |
| | NTY-amide |
| 15 | K(γE-C18diacid)K[CNTATC]ATQRL |
| | ANFLRHSSNN(αMePhe)GPILPPTEVGS |
| | NTY-amide |
| 16 | K(γE-C18diacid)[CNTATC]ATQRLA |
| | NFLRHSSNN(αMePhe)GPILPPTEVGSN |
| | TY-amide |
| 17 | K(O2Oc-O2Oc-γE-C18diacid) |
| | [CNTATC]ATQRLANFLRHSSNN |
| | (αMePhe)GPILPPTEVGSNTY-amide |
| 18 | K(γE-γE-C18diacid)[CNTATC]ATQ |
| | RLANFLRHSSNN(αMePhe)GPILPPTEV |
| | GSNTY-amide |
| 19 | K(γE-C18diacid)[CNTATC]ATQRLA |
| | NFLRHSSNN(αMePhe)GPILPPTNVGSN |
| | TY-amide |
| 20 | K(γE-γE-C18diacid)[CNTATC]ATQ |
| | RLANFLRHSSNN(αMePhe)GPILPPTNV |
| | GSNTY-amide |
| 21 | K(γE-γE-C18diacid)[CNTATC]ATQ |
| | RLANFLVHSSNN(αMePhe)GPILPPTNV |
| | GSRTY-amide |
| 22 | K(γE-γE-C18diacid)[CNTATC]ATQ |
| | RLANFLVHS(αMeSer)NNFGPILPPTNV |
| | GSNTY-amide |
| 23 | K(γE-γE-C18diacid)[CNTATC]ATQ |
| | RLANFLVHSSNN(αMePhe)GPILPPTEV |
| | GSNTY-amide |
| 24 | K(γE-γE-C18diacid)[CNTATC]ATQ |
| | RLANFLVHSSNN(αMePhe)GPILPPTRV |
| | GSNTY-amide |
| 25 | K(γE-C18diacid)[CNTATC]ATQRLA |
| | NFLVHSSNN(αMePhe)GPILPPTRVGSN |
| | TY-amide |
| 26 | K(γE-γE-C18diacid)[CNTATC]ATQ |
| | RLANFL(Aib)HSSNN(αMePhe)GPILP |
| | PTNVGSNTY-amide |
| 27 | K(γE-γE-C18diacid)[CNIATC]ATQ |
| | RLANFLVHSS(Dab)NFGPILPPTNVGSR |
| | TY-amide |
| 28 | K(γE-C18diacid)K[CNTATC]ATQRL |
| | ANFLVHSS(Dab)NFGPILPPTEVGSNT |
| | Y-amide |
| 29 | K(γE-C18diacid)K[CNTATC]ATQRL |
| | ANFLVHSS(Dab)NFGPILPPTNVGSNT |
| | Y-amide |
| 30 | K(γE-C18diacid)K[CNTATC]ATQRL |
| | ANFL(Aib)HSS(Dab)NFGPILPPTEVG |
| | SNTY-amide |
| 31 | K(γE-C18diacid)K[CNTATC]ATQRL |
| | ANFLVHSS(Dab)NFG(Aib)ILPPTNVG |
| | SNTY-amide |
| 32 | K(γE-C18diacid)K[CNTATC]ATQRL |
| | A(Dab)FLVHSSNN(αMePhe)GPILPPT |
| | EVGSNTY-amide |
| 33 | K(γE-C18diacid)K[CNTATC]ATQRL |
| | A(Dab)FLVHSSNNFGPILPPTNVGSNT |
| | Y-amide |
| 34 | K(γE-C18diacid)K[CNTATC]ATQRL |
| | A(Dab)FLVHSSNNFGPILPPTEVGSNT |
| | Y-amide |
| 35 | K(γE-C18diacid)K[CNTATC]ATQRL |
| | ANFLVHSS(Aib)NFGPILPPTNVGSNT |
| | Y-amide |
| 36 | K(γE-C18diacid)K[CNTATC]ATQRL |
| | ANFL(Aib)HSS(Aib)NFGPILPPTNVG |
| | SNTY-amide |
| 37 | K(γE-C18diacid)K[CNTATC]ATQRL |
| | ANFLSHSS(Aib)NFGPILPPTNVGSNT |
| | Y-amide |
| 38 | K(γE-C18diacid)K[CNTATC]ATQRL |
| | AEFLVHSS(Aib)NFGPILPPTNVGSNT |
| | Y-amide |
| 39 | K(γE-C18diacid)K[CNTATC]ATQRL |
| | ANFLEHSS(Aib)NFGPILPPTNVGSNT |
| | Y-amide |
| 40 | K(γE-C18diacid)K[CNTATC]ATQRL |
| | ANFLVHSS(Aib)NFGPILPPTHVGSNT |
| | Y-amide |
| 41 | K(γE-C18diacid)K[CNTATC]ATQRL |
| | ANFLVHSS(Aib)NFGPILPPTEVGSNT |
| | Y-amide |
| 42 | K(γE-C18diacid)K[CNTATC]ATQRL |
| | ANFLVHSS(Aib)NFGPILPPTNVGSET |
| | Y-amide |
| 43 | K(γE-C18diacid)K[CNTATC]ATQRL |
| | ANFLRHSS(Aib)NFGPILPPTEVGSNT |
| | Y-amide |
| 44 | K(γE-C18diacid)[CNTATC]ATQRLA |
| | NFLRHSS(Aib)NFGPILPPTEVGSNTY- |
| | amide |
| 45 | K(O2Oc-O2Oc-γE-C18diacid) |
| | [CNTATC]ATQRLANFLRHSS(Aib)NFG |
| | PILPPTEVGSNTY-amide |
| 46 | K(γE-C18diacid)K[CNTATC]ATQRL |
| | AHFLVHSS(Aib)NFGPILPPTNVGSNT |
| | Y-amide |
| 47 | K(γE-C18diacid)K[CNTATC]ATQRL |
| | AHFLVHSS(Aib)NFGPILPPTNVGSET |
| | Y-amide |
| 48 | K(γE-C18diacid)[CNTATC]ATQRLA |
| | NFLRHSS(Aib)NFGPILPPTNVGSNTY- |
| | amide |
| 49 | K(γE-γE-C18diacid)[CNTATC]ATQ |
| | RLANFLVHSS(Aib)NFGPILPPTEVGSN |
| | TY-amide |
| 50 | K(γE-γE-C18diacid)[CNTATC]ATQ |
| | RLANFLRHSS(Aib)NFGPILPPTEVGSN |
| | TY-amide |
| 51 | K(γE-C18diacid)K[CNTATC]ATQRL |
| | ANFLSHSS(Aib)NFGPILPPTHVGSNT |
| | Y-amide |
| 52 | K(γE-C18diacid)K[CNTATC]ATQRL |
| | ANFLSHSS(Aib)NFGPILPPTRVGSNT |
| | Y-amide |
| 53 | K(γE-C18diacid)K[CNTATC]ATQRL |
| | ANFLSHSS(Aib)NFGPILPPTPVGSNT |
| | Y-amide |
| 54 | K(γE-C18diacid)K[CNTATC]ATQRL |
| | ANFLSHSS(Aib)NFGPILPPTNVPSNT |
| | Y-amide |
| 55 | K(γE-C18diacid)K[CNTATC]ATQRL |
| | ANFLSHSS(Aib)NFGPILPPTNVGSPT |
| | Y-amide |
| 56 | K(γE-C18diacid)[CNTATC]ATQRLA |
| | NFLVHSS(Aib)NFGPILPPTNVGSNTY- |
| | amide |
| 57 | K(γE-γE-C18diacid)K[CNTATC]AT |
| | QRLANFLVHSS(Aib)NFGPILPPTNVGS |
| | NTY-amide |
| 58 | K(γE-C18diacid)K[CNTATC]ATQRL |
| | ANFLVHSS(Aib)NFGPILPPTNVGSNT |
| | P-amide |
| 59 | K(γE-C18diacid)[CNTATC]ATQRLA |
| | NFLVHSS(Aib)NFGPI(dL)PPTNVGSN |
| | TY-amide |
| 60 | K(γE-C18diacid)[CNTATC]ATQRLA |
| | NFLVHSS(Aib)NFGPIL(dP)PTNVGSN |
| | TY-amide |
| 61 | K(γE-C18diacid)[CNTATC]ATQRLA |
| | NFLVHSS(Aib)NFGP(dI)LPPTNVGSN |
| | TY-amide |
| 62 | K(γE-C18diacid)[CNTATC]ATQRLA |
| | NF(dL)VHSS(Aib)NFGPILPPTNVGSN |
| | TY-amide |
| 63 | K(γE-C18diacid)[CNTATC]ATQRLA |
| | NFLVHSS(Aib)NFGPILPPTRVGSNTY- |
| | amide |
| 64 | K(γE-C18diacid)[CNTATC]ATQRLA |
| | NFLVHSS(Aib)NFGPILPPTNVGSRTY- |
| | amide |
| 65 | K(γE-C18diacid)K[CNTATC]ATQRL |
| | ANFLRHSS(Aib)NFGPILPPTNVGSNT |
| | Y-amide |
| 66 | K(γE-γE-C18diacid)K[CNTATC]AT |
| | QRLANFLRHSS(Aib)NFGPILPPTNVGS |
| | NTY-amide |
| 67 | K(γE-γE-C18diacid)[CNTATC]ATQ |
| | RLANFLVHSS(Aib)NFGPILPPTRVGSN |
| | TY-amide |
| 68 | (C18diacid-γE-[CNTATC]ATQRLAN |
| | FLVHSS(Aib)NFGPILPPTRVGSNTY- |
| | amide |
| 69 | K(γE-C18diacid)K[CNTATC]ATQRL |
| | ANFLVHSS(Aib)NFGPILPPTRVGSNT |
| | Y-amide |
| 70 | K(γE-γE-C18diacid)K[CNTATC]AT |
| | QRLANFLVHSS(Aib)NFGPILPPTRVGS |
| | NTY-amide |
| 71 | C18diacid-γE-K[CNTATC]ATQRLAN |
| | FLVHSS(Aib)NFGPILPPTRVGSNTY- |
| | amide |
| 72 | K(γE-γE-C18diacid)[CNTATC]ATQ |
| | RLANFLVHSS(Aib)NFGPILPPTNVGSR |
| | TY-amide |
| 73 | K(γE-C18diacid)K[CNTATC]ATQRL |
| | ANFLVHSS(Aib)NFGPILPPTNVGSRT |
| | Y-amide |
| 74 | K(γE-γE-C18diacid)K[CNTATC]AT |
| | QRLANFLVHSS(Aib)NFGPILPPTNVGS |
| | RTY-amide |
| 75 | K(γE-γE-C18diacid)[CNTATC]ATQ |
| | RLANFLRHSS(Aib)NFGPILPPTNVGSN |
| | TY-amide |
| 76 | K(γE-C18diacid)[CNTATC]ATQRLA |
| | NFLRHSS(Aib)NFGPILPPTRVGSNTY- |
| | amide |
| 77 | K(γE-γE-C18diacid)[CNTATC]ATQ |
| | RLANFLRHSS(Aib)NFGPILPPTRVGSN |
| | TY-amide |
| 78 | K(γE-C18diacid)K[CNTATC]ATQRL |
| | ANFLVHSS(Aib)NFGP(Aib)LPPTNVG |
| | SNTY-amide |
| 79 | K(γE-C18diacid)K[CNTATC]ATQRL |
| | ANFLVHSS(Aib)NFGPI(Aib)PPTNVG |
| | SNTY-amide |
| 80 | K(γE-C18diacid)K[CNTATC]ATQRL |
| | ANFLVHSS(Aib)NFGPILPPT(Aib)VG |
| | SNTY-amide |
| 81 | K(γE-C18diacid)K[CNTATC]ATQRL |
| | ANFLVHSS(Aib)NFGPILPPTNV(Aib) |
| | SNTY-amide |
| 82 | K(γE-C18diacid)K[CNTATC]ATQRL |
| | ANFLVHSS(Aib)NFGPILPPTNVGS |
| | (Aib)TY-amide |
| 83 | K(γE-C18diacid)K[CNTATC]ATQRL |
| | ANFLVHSS(Aib)NFGPILPPTNVGSN |
| | (Aib)Y-amide |
| 84 | K(γE-C18diacid)K[CNTATC]ATQRL |
| | ANFLVHSS(Aib)NFGPILPPTNVG |
| | (Aib)NTY-amide |
| 85 | K(γE-C18diacid)K[CNTATC]ATQRL |
| | ANFLVHSS(Aib)NFGPILPPTNVGSNT |
| | (Aib)-amide |
| 86 | K(γE-C18diacid)K[CNTATC]ATQRL |
| | AHFL(Aib)HSS(Aib)NFGPILPPTEVG |
| | SNTY-amide |
| 87 | K(γE-C18diacid)K[CNTATC]ATQRL |
| | ANFL(Aib)HSS(Aib)NFGPILPPTNVG |
| | SNTP-amide |
| 88 | K(γE-C18diacid)K[CNTATC]ATQRL |
| | ANFLVHSS(Aib)NFGPIL(Aib)PTNVG |
| | SNTY-amide |
| 89 | K(γE-γE-C18diacid)[CNTATC]ATQ |
| | RLANFLVHSS(Aib)NFGPILPPT(Aib) |
| | VGSNTY-amide |
| 90 | K(γE-γE-C18diacid)[CNTATC]ATQ |
| | RLANFLRHSS(Aib)NFGPILPPT(Aib) |
| | VGSNTY-amide |
| 91 | K(γE-γE-C18diacid)[CNTATC]ATQ |
| | RLANFLVHSS(Aib)NFGPILPPTNVGS |
| | (Aib)TY-amide |
| 92 | K(γE-γE-C18diacid)[CNTATC]ATQ |
| | RLANFLRHSS(Aib)NFGPILPPTNVGS |
| | (Aib)TY-amide |
| 93 | K(γE-C18diacid)K[CNTATC]ATQRL |
| | ANFL(Aib)HSS(Dab)NFG(Aib)ILPP |
| | TNVGSNTY-amide |
| 94 | K(γE-C18diacid)K[CNTATC]ATQRL |
| | ANFL(Aib)HSSNNFGP(Aib)LPPTNVG |
| | SNTY-amide |
| 95 | K(γE-C18diacid)K[CNTATC]ATQRL |
| | ANFL(Aib)HSSNNFGPI(Aib)PPTNVG |
| | SNTY-amide |
| 96 | K(γE-C18diacid)K[CNTATC]ATQRL |
| | ANFL(Aib)HSSNNFGPIL(Aib)PTNVG |
| | SNTY-amide |
| 97 | K(γE-C18diacid)K[CNTATC]ATQRL |
| | ANFL(Aib)HSSNNFGPILP(Aib)TNVG |
| | SNTY-amide |
| 98 | K(γE-C18diacid)K[CNTATC]ATQRL |
| | ANFL(Aib)HSSNNFGPILPPT(Aib)VG |
| | SNTY-amide |
| 99 | K(γE-C18diacid)K[CNTATC]ATQRL |
| | ANFL(Aib)HSSNNFGPILPPTN(Aib)G |
| | SNTY-amide |
| 100 | K(γE-C18diacid)K[CNTATC]ATQRL |
| | ANFL(Aib)HSSNNFGPILPPTNV(Aib) |
| | SNTY-amide |
| 101 | K(γE-C18diacid)K[CNTATC]ATQRL |
| | ANFL(Aib)HSSNNFGPILPPTNVG |
| | (Aib)NTY-amide |
| 102 | K(γE-C18diacid)K[CNTATC]ATQRL |
| | ANFL(Aib)HSSNNFGPILPPTNVGS |
| | (Aib)TY-amide |
| 103 | K(γE-C18diacid)[CNTATC]ATQRLA |
| | NFLVHSSNNFGPI(dL)PPTNVGSNTY- |
| | amide |
| 104 | K(γE-C18diacid)[CNTATC]ATQRLA |
| | NFLVHSSNNFGPIL(dP)PTNVGSNTY- |
| | amide |
| 105 | K(γE-C18diacid)K[CNTATC]ATQRL |
| | ANFLSHSS(Dab)NFG(Aib)ILPPTNVG |
| | SNTY-amide |
| 106 | K(γE-C18diacid)K[CNTATC]ATQRL |
| | ANFLVHSSNNFGP(Aib)LPPTNVGSNT |
| | Y-amide |
| 107 | K(γE-C18diacid)K[CNTATC]ATQRL |
| | ANFLRHSSNNFGP(Aib)LPPTNVGSNT |
| | Y-amide |
| 108 | K(γE-C18diacid)K[CNTATC]ATQRL |
| | ANFLVHSSNNFGPI(Aib)PPTNVGSNT |
| | Y-amide |
| 109 | K(γE-C18diacid)K[CNTATC]ATQRL |
| | ANFLVHSSN(Aib)FGPILPPTNVGSNT |
| | Y-amide |
| 110 | K(γE-C18diacid)K[CNTATC]ATQRL |
| | ANFLVHSSNNF(Aib)PILPPTNVGSNT |
| | Y-amide |
| 111 | K(γE-C18diacid)K[CNTATC]ATQRL |
| | ANFLVHSSNHFGPILPPTNVGSETY- |
| | amide |
| 112 | C20diacid-γE-K[CNTATC]ATQRLAN |
| | FLVHSSNNFGPILPPTNVGSNTY-amide |
| 113 | C20diacid-γE-O2Oc-O2Oc-K |
| | [CNTATC]ATQRLANFLVHSSNNFGPILP |
| | PTNVGSNTY-amide |
| 114 | K(γE-γE-C20diacid)[CNTATC]ATQ |
| | RLANFLVHSSNNFGPILPPTNVGSRTY- |
| | amide |
| 115 | K(γE-γE-C20diacid)[CNTATC]ATQ |
| | RLANFLVHSSPNFPAILSPTNVGSNTY- |
| | amide |
| 116 | K(γE-γE-C20diacid)[CNTATC]ATQ |
| | RLAEFLRHSSNNFGPILPPTNVGSNTY- |
| | amide |
| 117 | K(γE-γE-C20diacid)[CNTATC]ATQ |
| | RLANFLVHSS(Aib)NFPAILSPTNVGSN |
| | TY-amide |
| 118 | K(γE-γE-C20diacid)[CNIATC]ATQ |
| | RLANFLVHSIANFGPILPPTNVGSRTY- |
| | amide |
| 119 | K(γE-γE-C20diacid)[CNTATC]ATQ |
| | RLANFLVHSPPNFPAILSPTNVGSNTY- |
| | amide |
| 120 | K(γE-γE-C20diacid)[CNAATC]ATQ |
| | RLANWLVHSSPNFPAILSPTNVGSNTY- |
| | amide |
| 121 | K(γE-γE-C20diacid)[CNTATC]ATQ |
| | RLANFLVHSS(Aib)NF(Hyp)AILSPTN |
| | VGSNTY-amide |
| 122 | K(γE-γE-C18diacid)[CNTATC]ATQ |
| | RLANFLVHSS(Aib)NFPAILSPT(Dab) |
| | VGSNTY-amide |
| 123 | K(γE-γE-C20diacid)[CNTATC]ATQ |
| | RLANFLVHSS(Aib)NFPAILSPT(Dab) |
| | VGSNTY-amide |
| 124 | K(γE-γE-C18diacid)[CNTATC]ATQ |
| | RLANFLVHSS(Aib)NFPAILSPTNVGS |
| | (Dab)TY-amide |
| 125 | K(γE-γE-C20diacid)[CNTATC]ATQ |
| | RLANFLVHSS(Aib)NFPAILSPTNVGS |
| | (Dab)TY-amide |
| 126 | K(γE-C18diacid)K[CNTATC]ATQRL |
| | ANFL(Aib)HSS(Dab)NFGPILPPTNVG |
| | SNTY-amide |
| 127 | K(γE-γE-C18diacid)[CNTATC]ATQ |
| | RLANFL(Aib)HSSNNFGPILPPTNVGSN |
| | TY-amide |
| 128 | K(γE-C18diacid)K[CNTATC]ATQRL |
| | AEFL(Aib)HSSNNFGPILPPTNVGSNT |
| | Y-amide |
| 129 | K(γE-C18diacid)K[CNTATC]ATQRL |
| | ANFL(Aib)HSSNNFGPILPPTNVGSNT |
| | Y-amide |
| 130 | K(γE-C18diacid)K[CNTATC]ATQRL |
| | AEFL(Aib)HSSHNFGPILPPTNVGSNT |
| | Y-amide |
| 131 | K(γE-C18diacid)K[CNTATC]ATQRL |
| | ANFL(Aib)HSSHNFGPILPPTNVGSNT |
| | Y-amide |
| 132 | K(γE-C18diacid)K[CNTATC]ATQRL |
| | ANFL(Aib)HSSPNFGPILPPTNVGSNT |
| | Y-amide |
| 133 | K(γE-C18diacid)K[CNTATC]ATQRL |
| | ANFL(Aib)HSSSNFGPILPPTNVGSNT |
| | Y-amide |
| 134 | K(γE-C18diacid)K[CNTATC]ATQRL |
| | ANFL(Aib)HSSNNFGPILPPTPVGSNT |
| | Y-amide |
| 135 | K(γE-C18diacid)K[CNTATC]ATQRL |
| | ANFL(Aib)HSSNHFGPILPPTNVGSNT |
| | Y-amide |
| 136 | K(γE-C18diacid)K[CNTATC]ATQRL |
| | ANFL(Aib)HSSNNFGPILPPTNVGSNT |
| | P-amide |
| 137 | K(γE-C18diacid)K[CNTATC]ATQRL |
| | ANFL(Aib)HSSRNFGPILPPTNVGSNT |
| | Y-amide |
| 138 | K(γE-C18diacid)K[CNTATC]ATQRL |
| | ANFL(Aib)HSSPNFGPILPPTEVGSNT |
| | Y-amide |
| 139 | K(γE-C18diacid)K[CNTATC]ATQRL |
| | ANFL(Aib)HSSPNFGPILPPTNVGSET |
| | Y-amide |
| 140 | K(γE-C18diacid)K[CNTATC]ATQRL |
| | ANFL(Aib)HSSNNFGPILPPTEVGSNT |
| | Y-amide |
| 141 | K(γE-C18diacid)K[CNTATC]ATQRL |
| | ANFL(Aib)HSSNNFGPILPPTNVGSRT |
| | Y-amide |
| 142 | K(γE-C18diacid)K[CNTATC]ATQRL |
| | ANFL(Aib)HSSNNFGPILPPTNVGSET |
| | Y-amide |
| 143 | K(γE-C18diacid)GGK[CNTATC]ATQ |
| | RLANFL(Aib)HSSNNFGPILPPTNVGSN |
| | TY-amide |
| 144 | K(γE-C18diacid)[CNTATC]ATQRLA |
| | HFL(Aib)HSSNNFGPILPPTNVGSNTY- |
| | amide |
| 145 | K(γE-C18diacid)K[CNTATC]ATQRL |
| | ANFL(Aib)HSSNNFGPILPPTNVGHNT |
| | Y-amide |
| 146 | K(γE-C18diacid)K[CNTATC]ATQRL |
| | ANFL(Aib)HSSNNFGPILPPTHVGSET |
| | Y-amide |
| 147 | K(γE-C18diacid)K[CNTATC]ATQRL |
| | ANFL(Aib)HSSNHFGPILPPTNVGSET |
| | Y-amide |
| 148 | K(γE-C18diacid)K[CNTATC]ATQRL |
| | ANFL(Aib)HSSNNFGPILPPTNVGPNT |
| | Y-amide |
| 149 | K(γE-C18diacid)[CNTATC]ATQRLA |
| | NFL(Aib)HSSNNFGPILPPTNVGSNTY- |
| | amide |
| 150 | K(γE-C18diacid)[CNTATC]ATQRLA |
| | NFL(Aib)HSS(Dab)NFGPILPPTNVGS |
| | NTY-amide |
| 151 | K(γE-C18diacid)[CNTATC]ATQRLA |
| | NFL(Aib)HSSNNFGPI(dL)PPTNVGSN |
| | TY-amide |
| 152 | K(γE-C18diacid)K[CNTATC]ATQRL |
| | ANFL(Aib)HSSNNFGPRLPPTNVGSNT |
| | Y-amide |
| 153 | K(γE-C18diacid)K[CNTATC]ATQRL |
| | ANFL(Aib)HSSKNFGPILPPTNVGSNT |
| | Y-amide |
| 154 | K(γE-C18diacid)K[CNTATC]ATQRL |
| | ANFL(Aib)HSSGNFGPILPPTNVGSNT |
| | Y-amide |
| 155 | K(γE-C18diacid)[CNTATC]ATQRLA |
| | NFL(Aib)HSSNNFGPILPPTRVGSNTY- |
| | amide |
| 156 | K(γE-C18diacid)K[CNTATC]ATQRL |
| | A(Dab)FL(Aib)HSSNNFGPILPPTEVG |
| | SNTY-amide |
| 157 | (C18diacid)K[CNTATC]ATQRLANFL |
| | VHSS(Aib)NFGPILPPT(Aib)VGSNT |
| | Y-amide |
| 158 | K(C18diacid)[CNTATC]ATQRLANFL |
| | VHSS(Aib)NFGPILPPT(Aib)VGSNT |
| | Y-amide |

Table 1: The square bracket [ ] between the two cysteine residues (cys 2 and cys 7) indicate the presence of an intramolecular disulphide bridge.

### DETAILED DESCRIPTION

The present inventors have observed that pramlintide conjugated to an albumin binding moiety, such as a lipid, has poor stability (e.g. the fibril-forming tendency of pramlintide is increased) under conditions required for drug product formulation. The present invention is based, at least in part, on the finding that the polypeptides (e.g. lipidated polypeptides) described herein may exhibit improved stability (e.g. reduced or no fibrillation tendency) as compared to such pramlintide conjugates.

For example, the present inventors have found that when pramlintide is conjugated to a lipid to increase the half-life, the fibril-forming tendency also increases. Accordingly, the polypeptides (e.g. lipidated polypeptides) described herein may bring the benefit of extended half-life compared to pramlintide but without the fibril-forming tendency of alternative lipidated pramlintide analogues. Peptides disclosed here can be formulated in or chemically conjugated to e.g. a protein, polymeric drug carrier or advance drug delivery system that enhances the chemical stability and or physical stability and or the circulatory exposure of the therapeutic moiety. The present inventors have further found that the polypeptides (e.g. lipidated polypeptides) described herein may exhibit improved physical and/or chemical stability as compared to human amylin or pramlintide. Furthermore, the polypeptides (e.g. lipidated polypeptides) described herein may have similar or improved selectivity to human amylin (hAMYR) compared to pramlintide.

Throughout this specification, amino acid positions of the polypeptides (e.g. lipidated polypeptides) are numbered according to the corresponding position in pramlintide having the sequence set forth in SEQ ID NO. 1.

Throughout this specification, amino acids are referred to by their conventional three-letter or single-letter abbreviations (e.g. Ala or A for alanine, Arg or R for arginine, etc.). In the case of certain less common or non-naturally occurring amino acids (i.e. amino acids other than the 20 encoded by the standard mammalian genetic code), unless they are referred to by their full name, frequently employed three- or four-character codes are employed for residues thereof, including aMeSer ((S)-2-amino-2-methyl-3-phenylpropanoic acid), αMePhe ((S)-2-amino-2-methyl-3-phenylpropanoic acid), Aib (2-amino-2-methylpropanoic acid), Dab (2,4-diaminobutanoic acid) and γ-Glu (γ-glutamic acid).

In embodiments of any aspect of the invention, the polypeptides (e.g. lipidated polypeptides) of the invention are isolated polypeptides (e.g. isolated lipidated polypeptides).

### Albumin Binding Moiety

The polypeptides of the invention comprise at least one albumin binding moiety. Without being bound by theory, it is thought that the albumin binding moiety protects the polypeptide against clearance and degradation, thereby extending the half-life of the polypeptide. As used herein, “albumin binding moiety” refers to a compound that binds to albumin. Exemplary albumin binding moieties suitable for use in the polypeptides of the invention include lipids (e.g. a fatty acid derivative), albumin-binding peptides, albumin-binding proteins, or small molecule ligands that bind to albumin. Optionally, the albumin binding moiety is a lipid, e.g. a lipid described herein.

The polypeptides of the invention may comprise one or more albumin binding moiety (e.g. lipid), e.g. one, two or three albumin binding moieties. In preferred embodiments, the polypeptides of the invention comprise only one albumin binding moiety (e.g. lipid).

The albumin binding moiety (e.g. lipid) may be attached to an amino acid residue of the polypeptide. In some embodiments, the albumin binding moiety (e.g. lipid) is attached to the amino acid residue through a linker. In alternative embodiments, the albumin binding moiety (e.g. lipid) is directly attached to the amino acid residue without an intervening linker. The albumin binding moiety (e.g. lipid) may be attached to the amino acid residue via an ester, a sulfonyl ester, a thioester, an amide, an amine or a sulphonamide. Accordingly, it will be understood that the albumin binding moiety (e.g. lipid) or the linker includes an acyl group, a sulphonyl group, an N atom, an O atom or an S atom which forms part of the ester, sulphonyl ester, thioester, amide, amine or sulphonamide. Optionally, an acyl group in the albumin binding moiety (e.g. lipid) or the linker forms part of an amide or ester with the amino acid residue. Accordingly, in preferred embodiments the albumin binding moiety (e.g. lipid) is attached to an acylation site on the amino acid residue.

The albumin binding moiety (e.g. lipid) may be attached to any residue at position Xaa-4 to Xaa 37 (e.g. to the EN of a lysine residue) of the polypeptide. In some embodiments, the albumin binding moiety (e.g. lipid) is attached to the side chain of an amino acid residue in the polypeptide, for example to the EN of a lysine residue. In some embodiments, the albumin binding moiety (e.g. lipid) is attached to the N-terminus of the polypeptide, (e.g. to a lysine at the N-terminus of the polypeptide).

In some embodiments, the albumin binding moiety (e.g. lipid) is attached to the N-terminus of the polypeptide, (e.g. to a lysine at the N-terminus of the polypeptide). In some embodiments, the albumin binding moiety (e.g. lipid) is attached to the amino acid residue at Xaa-4, Xaa-3, Xaa-2, Xaa-1 or Xaa 1 (e.g. to the EN of a lysine residue at Xaa-4, Xaa-3, Xaa-2, Xaa-1 or Xaa 1). In preferred embodiments, the albumin binding moiety (e.g. lipid) is attached to Xaa-4, Xaa-1 or Xaa 1 (either to the N-terminus or to the side chain of Xaa-4, Xaa-1 or Xaa 1).

### Lipid

In preferred embodiments, the albumin binding moiety is a lipid. Accordingly, the polypeptides of the invention may comprise at least one lipid (referred to herein as “lipidated polypeptide”). Without being bound by theory, it is thought that the lipid acts as an albumin binding moiety and protects the polypeptide against clearance and degradation, thereby extending the half-life of the polypeptide. The lipid may also modulate the potency of the compound as an agonist to the amylin (calcitonin) receptor.

In some embodiments, the polypeptide comprises at least one lipidated amino acid residue. In some embodiments, the polypeptide comprises at least two lipidated amino acid residues. In preferred embodiments, the polypeptide contains only one lipidated amino acid residue. The lipid may be attached to an amino acid residue of the polypeptide. In some embodiments, the lipid is attached to the amino acid residue through a linker (referred to herein as “linker-lipid”). In alternative embodiments, the lipid is directly attached to the amino acid residue without an intervening linker. The lipid may be attached to the amino acid residue via an ester, a sulfonyl ester, a thioester, an amide, an amine or a sulphonamide. Accordingly, it will be understood that the lipid or the linker includes an acyl group, a sulphonyl group, an N atom, an O atom or an S atom which forms part of the ester, sulphonyl ester, thioester, amide, amine or sulphonamide. Optionally, an acyl group in the lipid or linker forms part of an amide or ester with the amino acid residue. Accordingly, in preferred embodiments the lipid is attached to an acylation site on the amino acid residue.

The lipid may be attached to any residue at position Xaa-4 to Xaa 37 (e.g. to the EN of a lysine residue) of the polypeptide. In some embodiments, the lipid is attached to the side chain of an amino acid residue in the polypeptide, for example to the EN of a lysine residue. In some embodiments, the lipid is attached to the N-terminus of the polypeptide, (e.g. to a lysine at the N-terminus of the polypeptide).

In some embodiments, the lipid is attached to the N-terminus of the polypeptide, (e.g. to a lysine at the N-terminus of the polypeptide). In some embodiments, the lipid is attached to the amino acid residue at Xaa-4, Xaa-3, Xaa-2, Xaa-1 or Xaa 1 (e.g. to the EN of a lysine residue at Xaa-4, Xaa-3, Xaa-2, Xaa-1 or Xaa 1). In preferred embodiments, the lipid is attached to Xaa-4, Xaa-1 or Xaa 1 (either to the N-terminus or to the side chain of Xaa-4, Xaa-1 or Xaa 1).

In embodiments of any aspect of the invention, the lipid may comprise a hydrocarbon chain having from 10 to 26 C atoms, e.g. from 14 to 24 C atoms, e.g. from 16 to 22 C atoms. For example, the hydrocarbon chain may contain 14, 15, 16, 17, 18, 19, 20, 21, 22, 23 or 24 C atoms. In preferred embodiments, the lipid has 18 to 20 C atoms. In particular, the lipid may have 18 C atoms or 20 C atoms. The hydrocarbon chain may be linear or branched, and may be saturated or unsaturated. Furthermore, it can include a functional group at the end of the lipophilic chain, e.g. a carboxylic acid group which may or may not be protected during synthesis.

Optionally, the lipid comprises a dicarboxylic acid. For example, the lipid may comprise C12diacid, C14diacid, C16diacid, C17diacid, C18diacid, C19diacid or C20diacid. In preferred embodiments, the lipid comprises C18diacid or C20diacid.

### Linker

The albumin binding moiety (e.g. lipid) may be attached to the polypeptide through a linker. In embodiments of any aspect of the invention, the linker may comprise one or more residues of any naturally occurring or non-naturally occurring amino acid. The linker may comprise a combination of residues, as single or repeating units. For example, the linker may comprise multiple combinations of residues, as single or repeating units, each of which may independently be a residue of Glu, γ-Glu, Lys, ε-Lys, Asp, β-Asp, Gaba, β-Ala (3-aminopropanoyl), O2Oc (2-(2-(2-aminoethoxy) ethoxy) acetic acid), PEG2 (3-(2-(2-aminoethoxy) ethoxy) propanoic acid), PEG4 (1-amino-3,6,9,12-tetraoxapentadecan-15-oic acid), PEG8 (1-amino-3,6,9,12,15,18,21,24-octaoxaheptacosan-27-oic acid, PEG12 (1-amino-3,6,9,12,15,18,21,24,27,30,33,36-dodecaoxanonatriacontan-39-oic acid). γ-Glu and β-Asp refer to amino acids where the alpha-amino group and the side chain carboxyl group participate in peptide bond formation. ε-Lys refers to an amino acid where the epsilon-amino and carboxyl group of lysine participate in peptide bond formation.

In some embodiments, the linker comprises a residue of γ-Glu, e.g. γGlu, γGlu-γGlu, γGlu-(O2Oc)-(O2Oc) or γGlu-(PEG2)-(PEG2). In some embodiments, the linker consists of γGlu, γGlu-γGlu, γGlu-(O2Oc)-(O2Oc) or γGlu-(PEG2)-(PEG2).

In some embodiments of any aspect of the invention, the polypeptide comprises any one of the linker and lipid combinations set forth in any one of the rows in Table 2.

The linker may be attached to the amino acid residue via an ester, a sulfonyl ester, a thioester, an amide, an amine or a sulphonamide. Accordingly it will be understood that optionally the linker includes an acyl group, a sulphonyl group, an N atom, an O atom or an S atom which forms part of the ester, sulphonyl ester, thioester, amide, amine or sulphonamide. Optionally, an acyl group in the linker forms part of an amide or ester with the amino acid residue. Accordingly, in preferred embodiments the linker is attached to an acylation site on the amino acid residue.

The linker may be attached to a site (e.g. an acylation site) at the N-terminus of the lipidated polypeptide or to the & amino group “EN” of a residue in the lipidated polypeptide, e.g. to EN of a lysine residue.

In some embodiments, the polypeptide comprises a combination of linker, lipid and acylation site set forth in any one of the rows of Table 2.

TABLE 2
Combinations of linker, lipid and polypeptide acylation site

| | | Acylation | |
|---|---|---|---|
| Lipid | Linker | site | Formula |
| C18diacid | γE-γE | N- terminal | 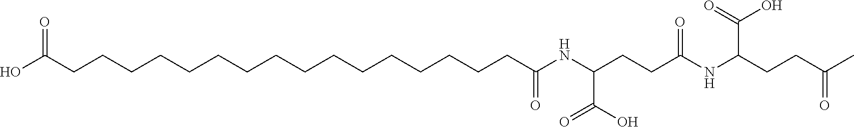 [SMILES: CC(=O)CCC(NC(=O)CCC(NC(=O)CCCCCCCCCCCCCCCCC(=O)O)C(=O)O)C(=O)O] |
| C18diacid | γE | (εN)K | 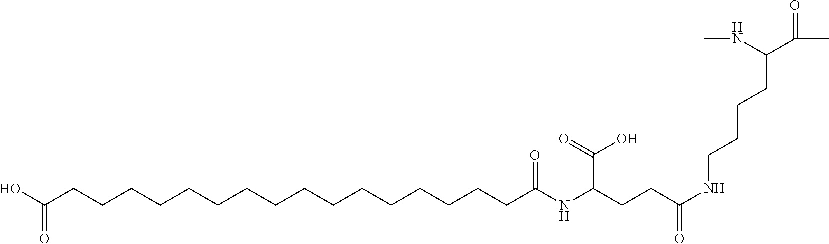 [SMILES: CNC(CCCCNC(=O)CCC(NC(=O)CCCCCCCCCCCCCCCCC(=O)O)C(=O)O)C(C)=O] |
| C18diacid | γE- (O2Oc)- (O2Oc) | (εN)K | 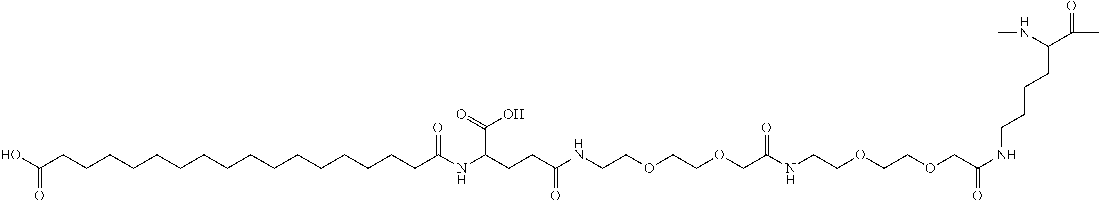 [SMILES: CNC(CCCCNC(=O)COCCOCCNC(=O)COCCOCCNC(=O)CCC(NC(=O)CCCCCCCCCCCCCCCCC(=O)O)C(=O)O)C(C)=O] |
| C18diacid | γE-γE | (εN)K | 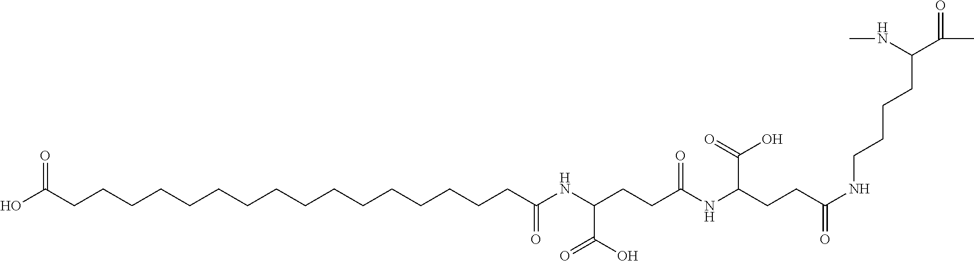 [SMILES: CNC(CCCCNC(=O)CCC(NC(=O)CCC(NC(=O)CCCCCCCCCCCCCCCCC(=O)O)C(=O)O)C(=O)O)C(C)=O] |
| C20diacid | γE-γE | N- terminal | 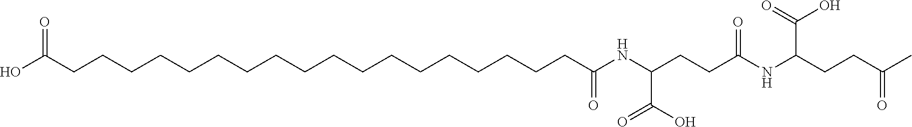 [SMILES: CC(=O)CCC(NC(=O)CCC(NC(=O)CCCCCCCCCCCCCCCCCCC(=O)O)C(=O)O)C(=O)O] |
| C20diacid | γE | N- terminal | 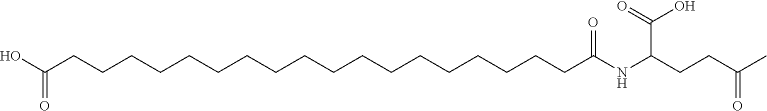 [SMILES: CC(=O)CCC(NC(=O)CCCCCCCCCCCCCCCCCCC(=O)O)C(=O)O] |
| C20diacid | γE- (O2Oc)- (O2Oc) | N- terminal | 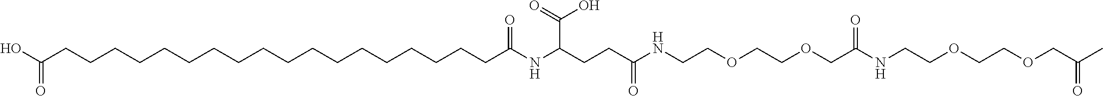 [SMILES: CC(=O)COCCOCCNC(=O)COCCOCCNC(=O)CCC(NC(=O)CCCCCCCCCCCCCCCCCCC(=O)O)C(=O)O] |
| C20diacid | γE-γE | (εN)K | 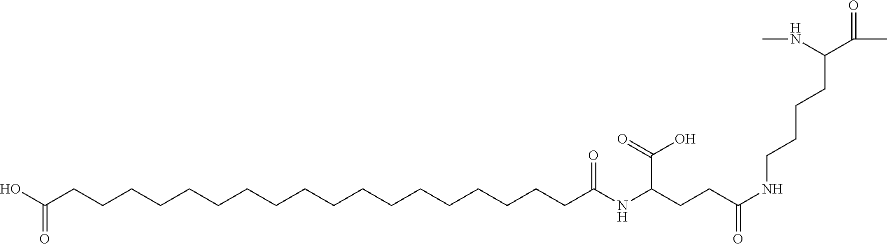 [SMILES: CNC(CCCCNC(=O)CCC(NC(=O)CCCCCCCCCCCCCCCCCCC(=O)O)C(=O)O)C(C)=O] |
| C18diacid | Nil | (εN)K | 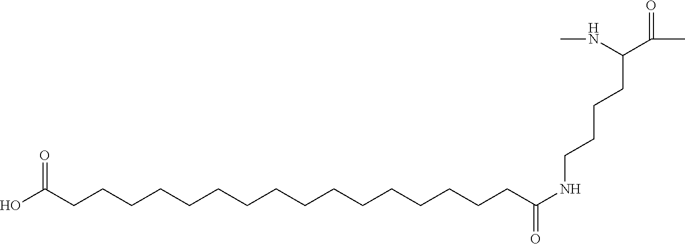 [SMILES: CNC(CCCCNC(=O)CCCCCCCCCCCCCCCCC(=O)O)C(C)=O] |
| C18diacid | Nil | N- terminal | 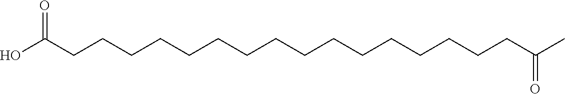 [SMILES: CC(=O)CCCCCCCCCCCCCCCCC(=O)O] |

The linker may be attached to any residue at position Xaa-4 to Xaa 37 (e.g. to the EN of a lysine residue) of the polypeptide. In some embodiments, the linker is attached to the side chain of an amino acid residue in the polypeptide, for example to the EN of a lysine residue. In some embodiments, the linker is attached to the N-terminus of the polypeptide, (e.g. to a lysine at the N-terminus of the polypeptide).

In some embodiments, the linker is attached to the N-terminus of the polypeptide, (e.g. to a lysine at the N-terminus of the polypeptide). In some embodiments, the linker is attached to the amino acid residue at Xaa-4, Xaa-3, Xaa-2, Xaa-1 or Xaa 1 (e.g. to the EN of a lysine residue at Xaa-4, Xaa-3, Xaa-2, Xaa-1 or Xaa 1). In preferred embodiments, the linker is attached to Xaa-4, Xaa-1 or Xaa 1 (either to the N-terminus or to the side chain of Xaa-4, Xaa-1 or Xaa 1).

In some embodiments, the linker is attached to a site (e.g. an acylation site) selected from the N-terminus of the polypeptide, EN of a lysine at position Xaa (1) “1K”, the EN of a lysine at position Xaa (−1) “−1K”, or the EN of a lysine at position Xaa (−4) “−4K”.

### Amino Acid Substitutions and Modifications

The polypeptides (e.g. lipidated polypeptides) of the invention may comprise one or more amino acid modifications or substitutions compared to the pramlintide sequence [SEQ ID NO: 1].

In some embodiments, the polypeptides (e.g. lipidated polypeptides) comprises one or more non-proteinogenic amino acids. Non-proteinogenic amino acids may include alpha methyl amino acids, D-enantiomers of naturally occurring amino acids, 2,4-diaminobutanoic acid (Dab), and (2S,4R)-4-hydroxypyrrolidine-2-carboxylic acid (Hyp). In some embodiments, the polypeptide (e.g. lipidated polypeptide) comprises one or more non-proteinogenic amino acids between positions 14-37, optionally at one or more of 14, 17 or 20-37.n some embodiments, the polypeptide (e.g. lipidated polypeptide) comprises one or more alpha methyl amino acids between positions 14-37, optionally at one or more of alpha methyl amino acids at positions 14, 17 or 20-37. Polypeptides (e.g. lipidated polypeptides) comprising one or more alpha methyl amino acids at positions 17, 21 or 23 are particularly preferred. Representative examples of alpha methyl amino acids include 2-amino-2-methylpropanoic acid (Aib), alpha-methyl glutamine (αMeGlu), alpha methyl phenylalanine (αMePhe or αMeF), alpha-methyl leucine (αMeLeu) and alpha-methyl serine (aMeSer). Thus, in certain embodiments, the alpha methyl amino acid can be Aib, αMeGlu, αMePhe, αMeLeu or αMeSer, or any combination thereof. In preferred embodiments, the polypeptide (e.g. lipidated polypeptide) comprises at least one alpha methyl amino acid, optionally selected from Aib, αMePhe and αMeSer. The reference to αMePhe and αMeF herein refers to(S)-2-amino-2-methyl-3-phenylpropanoic acid. The reference to αMeSer herein refers to(S)-2-amino-3-hydroxy-2-methylpropanoic acid. In preferred embodiments, the alpha methyl amino acid is Aib, αMePhe or αMeSer.

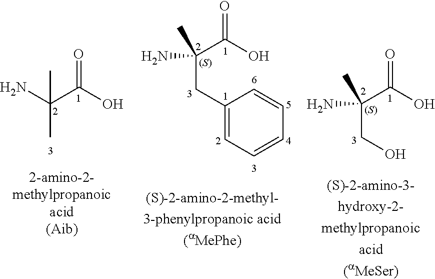

In some embodiments, the polypeptide (e.g. lipidated polypeptide) comprises one or more non-proteinogenic amino acids between positions 14-37 selected from the group consisting of: 2,4-diaminobutanoic acid (Dab), (2S,4R)-4-hydroxypyrrolidine-2-carboxylic acid (Hyp), D-leucine (dL), D-isoleucine (dl) and D-proline (dP).

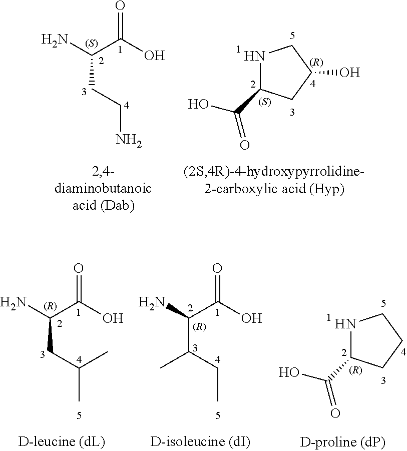

In some embodiments, the polypeptide (e.g. lipidated polypeptide) does not comprise (2S)-2-aminohexanedioic acid) (Aad) and/or does not comprise Aad at positions 14-37.

In some embodiments, the polypeptide (e.g. lipidated polypeptide) does not comprise Aib at one or more of positions 15, 16, 17, 19 or 20. In alternative embodiments, the polypeptide (e.g. lipidated polypeptide) comprises Aib at one or more of positions 15, 16, 17, 19 or 20 and at least one different non-proteinogenic amino acid (e.g. an alpha methyl amino acid that is not Aib) at positions 14-37.

In some embodiments, the polypeptide (e.g. lipidated polypeptide) comprises one or more natural amino acid substitutions or modifications compared to the pramlintide sequence [SEQ ID NO: 1].

In preferred embodiments, the polypeptide (e.g. lipidated polypeptide) comprises one or more of the following natural amino acid substitutions or modifications: deleted 1K(A1K), Ile 4, Ala 4, Glu 14, His 14, Trp 15, Arg 17, Ser 17, Glu 17, Pro 20, Ile 20, His 21, Ala 21, Glu 21, Gly 21, Lys 21, Pro 21, Arg 21, Ser 21, His 22, Pro 24, Ala 25, Arg 26, Ser 28, His 31, Glu 31, Pro 31, Arg 31, His 34, Pro 33, Pro 34, Glu 35, Arg 35, Pro 35 and Pro 37.

It will be understood that the polypeptide (e.g. lipidated polypeptides) may comprise a combination of non-proteinogenic amino acids and natural amino acid substitutions or modifications compared to the pramlintide sequence [SEQ ID NO: 1].

In some aspects, there is provided a polypeptide (e.g. lipidated polypeptide) that is a pramlintide analogue, or a pharmaceutically acceptable salt thereof, comprising any of the amino acid sequence modification combinations set forth in Table 3.

TABLE 3
Amino acid modifications to pramlintide sequence
Sequence modification with
respect to pramlintide
−1G, −2G, 17Aib
4A, 15W, 21P, 24P, 25A, 28S
4I, 20I, 21A, 35R
4I, 21Dab, 35R
14Dab
14Dab, 17Aib, 31E
14Dab, 23αMePhe, 31E
14Dab, 31E
14E, 17Aib
14E, 17Aib, 21H
14E, 17R
14E, 17R, 23αMePhe
14E, 21Aib
14H, 17Aib
14H, 17Aib, 21Aib, 31E
14H, 21Aib
14H, 21Aib, 35E
16dL, 21Aib
17Aib
17Aib, 37P
17Aib, 21Aib
17Aib, 21Aib, 37P
17Aib, 21G
17Aib, 21H
17Aib, 21K
17Aib, 21P
17Aib, 21P, 31E
17Aib, 21P, 35E
17Aib, 21R
17Aib, 21S
17Aib, 21Dab
17Aib, 21Dab, 31E
17Aib, 22H
17Aib, 22H, 35E
17Aib, 23αMePhe
17Aib, 26Aib
17Aib, 26R
17Aib, 27Aib
17Aib, 27dL
17Aib, 28Aib
17Aib, 29Aib
17Aib, 31Aib
17Aib, 31E
17Aib, 31H, 35E
17Aib, 31P
17Aib, 31R
17Aib, 32Aib
17Aib, 33Aib
17Aib, 34Aib
17Aib, 34H
17Aib, 34P
17Aib, 35Aib
17Aib, 35E
17Aib, 35R
17E, 21Aib
17R, 21Aib
17R, 21Aib, 31Aib
17R, 21Aib, 31E
17R, 21Aib, 31R
17R, 21Aib, 35Aib
17R, 23αMePhe
17R, 23αMePhe, 31E
17R, 26Aib
17S, 21Aib
17S, 21Aib, 31H
17S, 21Aib, 31P
17S, 21Aib, 31R
17S, 21Aib, 33P
17S, 21Aib, 35P
20αMeSer
20P, 21P, 24P, 25A, 28S
21Aib
21Aib, 24P, 25A, 28S
21Aib, 24P, 25A, 28S, 31Dab
21Aib, 24P, 25A, 28S, 35Dab
21Aib, 26dl
21Aib, 26Aib
21Aib, 27Aib
21Aib, 27dL
21Aib, 28Aib
21Aib, 28dP
21Aib, 31Aib
21Aib, 31E
21Aib, 31H
21Aib, 31R
21Aib, 33Aib
21Aib, 34Aib
21Aib, 35Aib
21Aib, 35E
21Aib, 35R
21Aib, 36Aib
21Aib, 37Aib
21Aib, 37P
21Aib, 24Hyp, 25A, 28S
21Dab, 24P, 25A, 28S
21Dab, 25Aib
21Dab, 31E
21P, 24P, 25A, 28S
22Aib
22H, 35E
23αMePhe
23αMePhe, 31E
23αMePhe, 31R
23αMePhe, 35R
24Aib
26Aib
27Aib
27dL
28dP
35R
Δ1K, 4I, 21Dab, 35R
Δ1K, 14E, 17R, 23αMePhe
Δ1K, 21Aib, 31R,

In one aspect, there is provided a polypeptide (e.g. lipidated polypeptide) that is a pramlintide analogue, or a pharmaceutically acceptable salt thereof, having an alpha methyl amino acid at position 23. In preferred embodiments, the alpha methyl amino acid is αMePhe.

In preferred embodiments of any aspect in which the polypeptide (e.g. lipidated polypeptide) comprises an alpha methyl amino acid (e.g. αMePhe) at position 23, the polypeptide (e.g. lipidated polypeptide) comprises any one of the following combinations of modifications:
14E, 17R, 23αMePhe;
Δ1K, 14E, 17R, 23αMePhe;
14Dab, 23αMePhe, 31E;
17Aib, 23αMePhe;
17R, 23αMePhe, 31E;
23αMePhe, 31E;
23αMePhe, 31R; or
23αMePhe, 35R.

In one aspect, there is provided a polypeptide (e.g. lipidated polypeptide) that is a pramlintide analogue, or a pharmaceutically acceptable salt thereof having at least two Aib residues. In preferred embodiments, the polypeptide (e.g. lipidated polypeptide) comprises Aib at at least two of positions 17 and 20-37. In particularly preferred embodiments, the polypeptide (e.g. lipidated polypeptide) comprises Aib at positions 21, 26, 27, 28, 29, 31, 32, 33, 34 and 35.

In preferred embodiments of any aspect in which the polypeptide (e.g. lipidated polypeptide) comprises at least two Aib residues, the polypeptide (e.g. lipidated polypeptide) comprises any one of the following combinations of modifications:
14H, 17Aib, 21Aib, 31E;
17Aib, 21Aib;
17Aib, 21Aib, 37P;
17Aib, 26Aib;
17Aib, 27Aib;
17Aib, 28Aib;
17Aib, 29Aib;
17Aib, 31Aib;
17Aib, 32Aib;
17Aib, 33Aib;
17Aib, 34Aib;
17Aib, 35Aib;
17R, 21Aib, 31Aib;
17R, 21Aib, 35Aib;
21Aib, 26Aib;
21Aib, 27Aib;
21Aib, 28Aib;
21Aib, 31Aib;
21Aib, 33Aib;
21Aib, 34Aib;
21Aib, 35Aib;
21Aib, 36Aib; or
21Aib, 37Aib.

In one aspect, there is provided a polypeptide (e.g. lipidated polypeptide) that is a pramlintide analogue, or a pharmaceutically acceptable salt thereof, having an alpha methyl amino acid at position 21. In preferred embodiments, the alpha methyl amino acid is Aib.

In preferred embodiments of any aspect in which the polypeptide (e.g. lipidated polypeptide) comprises an alpha methyl amino acid (e.g. Aib) at position 21, the polypeptide (e.g. lipidated polypeptide) comprises any one of the following combinations of modifications:
14E, 21Aib;
14H, 17Aib, 21Aib, 31E;
14H, 21Aib, 35E;
14H, 21Aib;
16dL, 21Aib;
17Aib, 21Aib, 37P;
17Aib, 21Aib;
17E, 21Aib;
17R, 21Aib, 31Aib;
17R, 21Aib, 31E;
17R, 21Aib, 31R;
17R, 21Aib, 35Aib;
17R, 21Aib;
17S, 21Aib, 31H;
17S, 21Aib, 31P;
17S, 21Aib, 31R;
17S, 21Aib, 33P;
17S, 21Aib, 35P;
17S, 21Aib;
21Aib, 24Hyp, 25A, 28S
21Aib, 24P, 25A, 28S, 31Dab;
21Aib, 24P, 25A, 28S, 35Dab.
21Aib, 24P, 25A, 28S;
21Aib, 26Aib;
21Aib, 26dl;
21Aib, 27Aib;
21Aib, 27dL;
21Aib, 28Aib;
21Aib, 28dP;
21Aib, 31Aib;
21Aib, 31E;
21Aib, 31H;
21Aib, 31R; A1K
21Aib, 33Aib;
21Aib, 34Aib;
21Aib, 35Aib;
21Aib, 35E;
21Aib, 35R;
21Aib, 36Aib;
21Aib, 37Aib;
21Aib, 37P; or
21Aib.

In one aspect, there is provided a polypeptide (e.g. lipidated polypeptide) that is a pramlintide analogue, or a pharmaceutically acceptable salt thereof, having an alpha methyl amino acid at position 17. In preferred embodiments, the alpha methyl amino acid is Aib.

In preferred embodiments of any aspect in which the polypeptide (e.g. lipidated polypeptide) comprises an alpha methyl amino acid (e.g. Aib) at position 17, the polypeptide (e.g. lipidated polypeptide) comprises any one of the following combinations of modifications:
14H, 17Aib;
−1G, −2G, 17Aib;
17Aib, 23αMePhe;
17Aib, 21Dab, 31E;
17Aib, 21Dab;
17Aib, 21Aib;
14H, 17Aib, 21Aib, 31E;
17Aib, 21Aib, 37P;
17Aib, 26Aib;
17Aib, 27Aib;
17Aib, 28Aib;
17Aib, 29Aib;
17Aib, 31Aib;
17Aib, 32Aib;
17Aib, 33Aib;
17Aib, 34Aib; or
17Aib, 35Aib.

### Pharmacokinetics

The polypeptides (e.g. lipidated polypeptides) of the invention may exhibit favourable pharmacokinetic properties as compared to pramlintide. For example, the polypeptides (e.g. lipidated polypeptides) of the invention may have an extended half-life as compared to pramlintide.

As used herein, the term “half-life” is used to refer to the time taken for the concentration of isolated polypeptide in plasma to decline to 50% of its original level. Methods to determine the half-life of proteins are known in the art and are described in Example 4.

It will be recognised that an extended half-life is advantageous, as it permits the therapeutic proteins to be administered according to a safe and convenient dosing schedule, e.g. lower doses that can be administered less frequently. Moreover, the achievement of lower doses may provide further advantages such as the provision of an improved safety profile. To the contrary, pramlintide requires frequent and inconvenient administration.

The present inventors have shown that the polypeptides (e.g. lipidated polypeptides) of the invention may have a half-life of at least 4 hours in rat models (see Example 4). In embodiments, the polypeptide (e.g. lipidated polypeptide) has a half-life of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours or at least 14 hours in rat models. In preferred embodiments, the polypeptide (e.g. lipidated polypeptide) has a half-life of at least 14 hours.

### Reduced Fibrillation

The polypeptides (e.g. lipidated polypeptides) of the invention may exhibit reduced tendency to undergo fibrillation in pharmaceutically relevant aqueous media, especially at pH values in the range from 4 to 7, as compared to lipidated pramlintide. In some embodiments, the polypeptide (e.g. lipidated polypeptide) exhibits reduced tendency to undergo fibrillation in pharmaceutically relevant aqueous media, especially at pH values in the range from 4 to 7, as compared to pramlintide which is lipidated in a similar manner e.g. the same lipid is attached, the lipid is attached through the same linker and/or the lipid is attached at the same position. Exemplary lipidated pramlintide molecules are given in Table 1, for example SEQ ID NO. 3, 4, 5, 6, 7, 112 and 113.

Accordingly, the polypeptides (e.g. lipidated polypeptides) of the invention may be suited for formulation in acidic media (e.g. pH 4) and in neutral or near-neutral media (e.g. pH 7 or 7.4). Such polypeptides (e.g. lipidated polypeptides) may be well suited for co-formulation with, for example, insulin, various insulin analogues and/or other therapeutic (e.g. anti-diabetic or anti-obesity) agents that require a neutral or near-neutral formulation pH.

In some embodiments, the polypeptide (e.g. lipidated polypeptide) shows no detectable fibrillation after about 5 hours, after about 7 hours, after about 9 hours, after about 11 hours, after about 13 hours, after about 15 hours, after about 17 hours or after about 20 hours at pH 4 and 37° C., e.g. under the conditions described in Example 3.

In preferred embodiments, the polypeptide (e.g. lipidated polypeptide) shows no detectable fibrillation after about 48 hours, after about 72 hours, after about 96 hours, after about 108 hours, after about 120 hours, after 132 about hours or after about 144 hours at pH 4 and 37° C., e.g. under the conditions described in Example 3. In particularly preferred embodiments, the polypeptide (e.g. lipidated polypeptide) shows no detectable fibrillation after 144 hours at pH 4 and 37° C., e.g. under the conditions described in Example 3.

In some embodiments, the formation of fibrils is detected by an increase in fluorescence intensity in a Thioflavin T fibrillation assay, e.g. as described in Example 3.

In preferred embodiments, the polypeptides (e.g. lipidated polypeptides) of the invention are soluble at concentrations required for therapeutic efficacy. In some embodiments, the lipidated polypeptides of the invention are soluble at a concentration of at least 1 mg/mL under the conditions described in Example 3.

### Efficacy

The polypeptides (e.g. lipidated polypeptides) of the invention are amylin receptor agonists, i.e. they are capable of binding to, and inducing signalling by, one or more receptors or receptor complexes regarded as physiological receptors for human amylin. These include the human calcitonin receptor hCTR, as well as complexes comprising the human calcitonin receptor hCTR and at least one of the human receptor activity modifying proteins designated hRAMP1, hRAMP2 and hRAMP3. Complexes between hCTR and hRAMP1, hRAMP2 and hRAMP3 are designated hAMYR1, hAMYR2 and hAMYR3 (i.e. human amylin receptors 1, 2 and 3) respectively. In some embodiments, a compound is considered an amylin receptor agonist if it has agonist activity at one or more of hAMYR1, hAMYR2 and hAMYR3. For example, a compound may be considered an amylin receptor agonist if it has agonist activity at hAMYR3.

The ability to induce CAMP formation as a result of binding to the relevant receptor or receptor complex is typically regarded as indicative of agonist activity. Other intracellular signaling pathways or events may also be used as readouts for amylin receptor agonist activity. These may include calcium release, arrestin recruitment, receptor internalization, kinase activation or inactivation, lipase activation, inositol phosphate release, diacylglycerol release or nuclear transcription factor translocation.

EC50 values may be used as a measure of agonist potency at a given receptor. An EC50 value is a measure of the concentration of a compound required to achieve half of that compound's maximal activity in a particular assay, for example a CAMP assay as described in Example 2. In Example 2, the present inventors have shown that certain polypeptides (e.g. lipidated polypeptides) disclosed herein exhibit greater or similar selectivity to hAMYR over hCTR as pramlintide, as measured using CAMP release from binding to hAMYR and hCTR. Pramlintide exhibits at least 10-fold selectivity to hAMYR as compared to hCTR.

The polypeptides (e.g. lipidated polypeptides) of the invention may exhibit improved efficacy, e.g. as amylin receptor agonists, as compared to lipidated pramlintide.

In some embodiments, the polypeptide (e.g. lipidated polypeptide) has at least about 1-fold selectivity to hAMYR over hCTR, optionally at least about 2-fold, at least about 4-fold, at least about 6-fold, at least about 8-fold, at least about 10-fold, at least about 12-fold, at least about 14-fold, at least about 16-fold, at least about 18-fold, at least about 20-fold, at least about 50-fold, at least about 75-fold, or at least about 100-fold selectivity to hAMYR over hCTR. In preferred embodiments, the polypeptide (e.g. lipidated polypeptide) has at least about 10-fold selectivity to hAMYR over hCTR.

In some embodiments, the polypeptide (e.g. lipidated polypeptide) has around 12-20 fold, around 14-18 fold, optionally around 16-fold selectivity to hAMYR over hCTR.

n some embodiments, the isolated polypeptide has an EC50 measured under the conditions described in Example 2 (i.e. containing 0.1% bovine serum albumin (BSA)) of below about 1.4 nM, below about 1.2 nM, below about 1 nM, below about 0.8 nM, below about 0.6 nM, below about 0.4 nM, below about 0.3 nM, or below about 0.2 nM.

### Chemical Stability

The polypeptides (e.g. lipidated polypeptides) of the invention may be chemically stable, e.g. they may form in a formulation an acceptable percentage of degradation products produced over a defined period of time by chemical pathways, such as deamidation, aggregation, or oxidation.

The polypeptides (e.g. lipidated polypeptides) of the invention may be chemically conjugated to a protein or polymeric drug carrier, or formulated in an advance drug delivery system, that enhances the chemical stability and/or physical stability and/or the circulatory exposure of the polypeptide.

In some aspects, there is provided a polypeptide or a pharmaceutically acceptable salt thereof, wherein the polypeptide comprises any one of the lipid linkers as set forth in Table 2 and any one of the sequence modifications as set forth in Table 3.

In some aspects, there is provided a polypeptide or a pharmaceutically acceptable salt thereof, wherein the polypeptide comprises the lipid linker and amino acid sequence modification combinations set forth in Table 4.

TABLE 4
Lipidated polypeptides

| SEQ | | | Acyl | Sequence modification |
|---|---|---|---|---|
| ID | | | tion | with respect |
| NO: | Lipid | Linker | site | to pramlintide |
| 8 | C18diacid | γE-γE | 1K | 21Dab, 24P, 25A, 28S |
| 9 | C18diacid | γE-γE | 1K | 21Aib, 24P, 25A, 28S |
| 10 | C18diacid | γE | N- | 14E, 17R, |
| | | | terminal | 23αMePhe, an dΔ1K |
| 11 | C18diacid | γE-γE | 1K | 14E, 17R, 23αMePhe |
| 12 | C18diacid | γE | −1K | 14E, 17R, 23αMePhe |
| 13 | C18diacid | γE-γE | 1K | 23αMePhe |
| 14 | C18diacid | γE | −1K | 23αMePhe |
| 15 | C18diacid | γE | −1K | 17R, 23αMePhe, 31E |
| 16 | C18diacid | γE | 1K | 17R, 23αMePhe, 31E |
| 17 | C18diacid | γE-(O2Oc)- | 1K | 17R, 23αMePhe, 31E |
| | | (O2Oc) | | |
| 18 | C18diacid | γE-γE | 1K | 17R, 23αMePhe, 31E |
| 19 | C18diacid | γE | 1K | 17R, 23αMePhe |
| 20 | C18diacid | γE-γE | 1K | 17R, 23αMePhe |
| 21 | C18diacid | γE-γE | 1K | 23αMePhe, 35R |
| 22 | C18diacid | γE-γE | 1K | 20αMeS |
| 23 | C18diacid | γE-γE | 1K | 23αMePhe, 31E |
| 24 | C18diacid | γE-γE | 1K | 23αMePhe, 31R |
| 25 | C18diacid | γE | 1K | 23αMePhe, 31R |
| 26 | C18diacid | γE-γE | 1K | 17Aib, 23αMePhe |
| 27 | C18diacid | γE-γE | 1K | 4I, 21Dab, 35R |
| 28 | C18diacid | γE | −1K | 21Dab, 31E |
| 29 | C18diacid | γE | −1K | 21Dab |
| 30 | C18diacid | γE | −1K | 17Aib, 21Dab, 31E |
| 31 | C18diacid | γE | −1K | 21Dab, 25Aib |
| 32 | C18diacid | γE | −1K | 14Dab, 23αMePhe, 31E |
| 33 | C18diacid | γE | −1K | 14Dab |
| 34 | C18diacid | γE | −1K | 14Dab, 31E |
| 35 | C18diacid | γE | −1K | 21Aib |
| 36 | C18diacid | γE | −1K | 17Aib, 21Aib |
| 37 | C18diacid | γE | −1K | 17S, 21Aib |
| 38 | C18diacid | γE | −1K | 14E, 21Aib |
| 39 | C18diacid | γE | −1K | 17E, 21Aib |
| 40 | C18diacid | γE | −1K | 21Aib, 31H |
| 41 | C18diacid | γE | −1K | 21Aib, 31E |
| 42 | C18diacid | γE | −1K | 21Aib, 35E |
| 43 | C18diacid | γE | −1K | 17R, 21Aib, 31E |
| 44 | C18diacid | γE | 1K | 17R, 21Aib, 31E |
| 45 | C18diacid | γE-(O2Oc)- | 1K | 17R, 21Aib, 31E |
| | | (O2Oc) | | |
| 46 | C18diacid | γE | −1K | 14H, 21Aib |
| 47 | C18diacid | γE | −1K | 14H, 21Aib, 35E |
| 48 | C18diacid | γE | 1K | 17R, 21Aib |
| 49 | C18diacid | γE-γE | 1K | 21Aib, 31E |
| 50 | C18diacid | γE-γE | 1K | 17R, 21Aib, 31E |
| 51 | C18diacid | γE | −1K | 17S, 21Aib, 31H |
| 52 | C18diacid | γE | −1K | 17S, 21Aib, 31R |
| 53 | C18diacid | γE | −1K | 17S, 21Aib, 31P |
| 54 | C18diacid | γE | −1K | 17S, 21Aib, 33P |
| 55 | C18diacid | γE | −1K | 17S, 21Aib, 35P |
| 56 | C18diacid | γE | 1K | 21Aib |
| 58 | C18diacid | γE | −1K | 21Aib, 37P |
| 59 | C18diacid | γE | 1K | 21Aib, 27dL |
| 60 | C18diacid | γE | 1K | 21Aib, 28dP |
| 61 | C18diacid | γE | 1K | 21Aib, 26dl |
| 62 | C18diacid | γE | 1K | 16dL, 21Aib |
| 63 | C18diacid | γE | 1K | 21Aib, 31R |
| 64 | C18diacid | γE | 1K | 21Aib, 35R |
| 65 | C18diacid | γE | −1K | 17R, 21Aib |
| 66 | C18diacid | γE-γE | −1K | 17R, 21Aib |
| 67 | C18diacid | γE-γE | 1K | 21Aib, 31R |
| 68 | C18diacid | γE | N- | 21Aib, 31R, Δ1K |
| | | | terminal | |
| 69 | C18diacid | γE | −1K | 21Aib, 31R |
| 70 | C18diacid | γE-γE | −1K | 21Aib, 31R |
| 71 | C18diacid | γE | −1K | 21Aib, 31R |
| 72 | C18diacid | γE-γE | 1K | 21Aib, 35R |
| 73 | C18diacid | γE | −1K | 21Aib, 35R |
| 74 | C18diacid | γE-γE | −1K | 21Aib, 35R |
| 75 | C18diacid | γE-γE | 1K | 17R, 21Aib |
| 76 | C18diacid | γE | 1K | 17R, 21Aib, 31R |
| 77 | C18diacid | γE-γE | 1K | 17R, 21Aib, 31R |
| 78 | C18diacid | γE | −1K | 21Aib, 26Aib |
| 79 | C18diacid | γE | −1K | 21Aib, 27Aib |
| 80 | C18diacid | γE | −1K | 21Aib, 31Aib |
| 81 | C18diacid | γE | −1K | 21Aib, 33Aib |
| 82 | C18diacid | γE | −1K | 21Aib, 35Aib |
| 83 | C18diacid | γE | −1K | 21Aib, 36Aib |
| 84 | C18diacid | γE | −1K | 21Aib, 34Aib |
| 85 | C18diacid | γE | −1K | 21Aib, 37Aib |
| 86 | C18diacid | γE | −1K | 14H, 17Aib, 21Aib, 31E |
| 87 | C18diacid | γE | −1K | 17Aib, 21Aib, 37P |
| 88 | C18diacid | γE | −1K | 21Aib, 28Aib |
| 89 | C18diacid | γE-γE | 1K | 21Aib, 31Aib |
| 90 | C18diacid | γE-γE | 1K | 17R, 21Aib, 31Aib |
| 91 | C18diacid | γE-γE | 1K | 21Aib, 35Aib |
| 92 | C18diacid | γE-γE | 1K | 17R, 21Aib, 35Aib |
| 94 | C18diacid | γE | −1K | 17Aib, 26Aib |
| 95 | C18diacid | γE | −1K | 17Aib, 27Aib |
| 96 | C18diacid | γE | −1K | 17Aib, 28Aib |
| 97 | C18diacid | γE | −1K | 17Aib, 29Aib |
| 98 | C18diacid | γE | −1K | 17Aib, 31Aib |
| 99 | C18diacid | γE | −1K | 17Aib, 32Aib |
| 100 | C18diacid | γE | −1K | 17Aib, 33Aib |
| 101 | C18diacid | γE | −1K | 17Aib, 34Aib |
| 102 | C18diacid | γE | −1K | 17Aib, 35Aib |
| 103 | C18diacid | γE | 1K | 27dL |
| 104 | C18diacid | γE | 1K | 28dP |
| 106 | C18diacid | γE | −1K | 26Aib |
| 107 | C18diacid | γE | −1K | 17R, 26Aib |
| 108 | C18diacid | γE | −1K | 27Aib |
| 109 | C18diacid | γE | −1K | 22Aib |
| 110 | C18diacid | γE | −1K | 24Aib |
| 111 | C18diacid | γE | −1K | 22H, 35E |
| 114 | C20diacid | γE-γE | 1K | 35R |
| 115 | C20diacid | γE-γE | 1K | 21P, 24P, 25A, 28S |
| 116 | C20diacid | γE-γE | 1K | 14E, 17R |
| 117 | C20diacid | γE-γE | 1K | 21Aib, 24P, 25A, 28S |
| 118 | C20diacid | γE-γE | 1K | 4I, 201, 21A, 35R |
| 119 | C20diacid | γE-γE | 1K | 20P, 21P, 24P, 25A, |
| | | | | 28S |
| 120 | C20diacid | γE-γE | 1K | 4A, 15W, 21P, 24P, |
| | | | | 25A, 28S |
| 121 | C20diacid | γE-γE | 1K | 21Dab, 24Hyp, 25A, |
| | | | | 28S |
| 122 | C18diacid | γE-γE | 1K | 21Aib, 24P, 25A, |
| | | | | 28S, 31Dab |
| 123 | C20diacid | γE-γE | 1K | 21Aib, 24P, 25A, |
| | | | | 28S, 31Dab |
| 124 | C18diacid | γE-γE | 1K | 21Aib, 24P, 25A, |
| | | | | 28S, 35Dab |
| 125 | C20diacid | γE-γE | 1K | 21Aib, 24P, 25A, |
| | | | | 28S, 35Dab |
| 126 | C18diacid | γE | −1K | 17Aib, 21Dab |
| 127 | C18diacid | γE-γE | 1K | 17Aib |
| 128 | C18diacid | γE | −1K | 14E, 17Aib |
| 129 | C18diacid | γE | −1K | 17Aib |
| 130 | C18diacid | γE | −1K | 14E, 17Aib, 21H |
| 131 | C18diacid | γE | −1K | 17Aib, 21H |
| 132 | C18diacid | γE | −1K | 17Aib, 21P |
| 133 | C18diacid | γE | −1K | 17Aib, 21S |
| 134 | C18diacid | γE | −1K | 17Aib, 31P |
| 135 | C18diacid | γE | −1K | 17Aib, 22H |
| 136 | C18diacid | γE | −1K | 17Aib, 37P |
| 137 | C18diacid | γE | −1K | 17Aib, 21R |
| 138 | C18diacid | γE | −1K | 17Aib, 21P, 31E |
| 139 | C18diacid | γE | −1K | 17Aib, 21P, 35E |
| 140 | C18diacid | γE | −1K | 17Aib, 31E |
| 141 | C18diacid | γE | −1K | 17Aib, 35R |
| 142 | C18diacid | γE | −1K | 17Aib, 35E |
| 143 | C18diacid | γE | −3K | −1G, −2G, 17Aib |
| 144 | C18diacid | γE | 1K | 14H, 17Aib |
| 145 | C18diacid | γE | −1K | 17Aib, 34H |
| 146 | C18diacid | γE | −1K | 17Aib, 31H, 35E |
| 147 | C18diacid | γE | −1K | 17Aib, 22H, 35E |
| 148 | C18diacid | γE | −1K | 17Aib, 34P |
| 149 | C18diacid | γE | 1K | 17Aib |
| 150 | C18diacid | γE | 1K | 17Aib, 21Dab |
| 151 | C18diacid | γE | 1K | 17Aib, 27dL |
| 152 | C18diacid | γE | −1K | 17Aib, 26R |
| 153 | C18diacid | γE | −1K | 17Aib, 21K |
| 154 | C18diacid | γE | −1K | 17Aib, 21G |
| 155 | C18diacid | γE | 1K | 17Aib, 31R |
| 156 | C18diacid | γE | −1K | 14Dab, 17Aib, 31E |
| 157 | C18diacid | Nil | N- | 21Aib, 31Aib |
| | | | terminal | |
| 158 | C18diacid | Nil | 1K | 21Aib, 31Aib |

### Process

The polypeptides (e.g. lipidated polypeptides) of the invention may be produced by any method known in the art. The production of polypeptides such as amylin or analogues thereof is well known in the art. The polypeptide (e.g. lipidated polypeptides) of the invention can thus be produced by chemical synthesis, e.g. solid phase polypeptide synthesis using t-Boc or Fmoc chemistry, or other well-established techniques. They may alternatively be produced by recombinant expression of a nucleic acid molecule encoding a fusion polypeptide in a host cell. Following synthesis, the polypeptides (e.g. lipidated polypeptides) of the invention may optionally be isolated or purified.

### Therapeutic Methods

In further aspects, the polypeptides (e.g. lipidated polypeptides) of the invention are provided in a pharmaceutical composition.

The pharmaceutical compositions of the invention may comprise one or more excipient(s). Pharmaceutically acceptable excipients are known in the art, see for instance Remington's Pharmaceutical Sciences (by Joseph P. Remington, 18th ed., Mack Publishing Co., Easton, PA), which is incorporated herein in its entirety.

The present invention encompasses therapies which involve administering the polypeptides (e.g. lipidated polypeptides) of the invention to an animal, in particular a mammal, for instance a human, for preventing, treating, or ameliorating symptoms associated with a disease, disorder, or infection.

Accordingly, the polypeptides (e.g. lipidated polypeptides) or pharmaceutical compositions of the invention may be used in therapy, for example for treating a disease or disorder. Also provided is a method of treating a disease or disorder comprising administering to a subject or patient in need thereof a therapeutically effective amount of the polypeptides (e.g. lipidated polypeptides) or pharmaceutical compositions of the invention. The use or method may comprise administering a therapeutically effective schedule that has less frequent doses of the polypeptides (e.g. lipidated polypeptides) of the invention than the therapeutically effective dosing schedule of pramlintide.

It will be understood that the polypeptides (e.g. lipidated polypeptides) of the invention may be used in the treatment and/or prevention of obesity, metabolic diseases such as diabetes (e.g. type 1 or type 2 diabetes), and/or obesity-related conditions.

Accordingly, the polypeptides (e.g. lipidated polypeptides) of the invention may be used in a method of treating obesity, overweight, morbid obesity, obesity prior to surgery, obesity-linked inflammation, obesity-linked gallbladder disease, sleep apnea and respiratory problems, hyperlipidemia, degeneration of cartilage, osteoarthritis, or reproductive health complications of obesity or overweight such as infertility in a subject, the method comprising administering a therapeutically effective amount of the polypeptide (e.g. lipidated polypeptide) to the subject.

This is also provided a method of inhibiting or reducing weight gain, promoting weight loss, reducing food intake, and/or reducing excess body weight, the method comprising administering the polypeptide (e.g. lipidated polypeptide) of the invention to the subject.

Metabolic diseases that may be treated by the polypeptide (e.g. lipidated polypeptide) of the invention include diabetes, type 1 diabetes, type 2 diabetes, gestational diabetes, pre-diabetes, insulin resistance, impaired glucose tolerance (IGI), disease states associated with elevated blood glucose levels, metabolic disease including metabolic syndrome, or hyperglycemia e.g. abnormal postprandial hyperglycemia.

In preferred embodiments, the polypeptides (e.g. lipidated polypeptides) of the invention are used for the treatment of type 1 diabetes or type 2 diabetes.

The polypeptides (e.g. lipidated polypeptides) or pharmaceutical compositions of the invention may be used for treating, inhibiting or reducing weight gain, promoting weight loss, reducing food intake, and/or reducing excess body weight.

The polypeptides (e.g. lipidated polypeptides) or pharmaceutical compositions of the invention may be used in the treatment and/or prevention of an eating disorder, Alzheimer's disease, hepatic steatosis (“fatty liver”), kidney failure, arteriosclerosis (e.g. atherosclerosis), cardiovascular disease, macrovascular disease, microvascular disease, diabetic heart (including diabetic cardiomyopathy and heart failure as a diabetic complication), coronary heart disease, peripheral artery disease or stroke, cancer, dumping syndrome, hypertension e.g. pulmonary hypertension, or dyslipidemia e.g. atherogenic dyslipidemia, cholescystitis, or short bowel syndrome.

The route of administration of polypeptides (e.g. lipidated polypeptides) of the invention, or pharmaceutical compositions thereof, can be, for example, oral, parenteral, by inhalation or topical. In preferred embodiments, the polypeptide (e.g. lipidated polypeptide) or pharmaceutical composition thereof is administered by parenteral administration to a subject or patient. The term “parenteral” as used herein includes, e.g., intravenous, intraarterial, intraperitoneal, intramuscular, subcutaneous, rectal, or vaginal administration. In preferred embodiments, the polypeptide (e.g. lipidated polypeptide) or pharmaceutical composition thereof is administered by injection, such as by intravenous, subcutaneous or intramuscular injection, to a subject or patient. In particularly preferred embodiments, the polypeptide (e.g. lipidated polypeptide) or pharmaceutical composition thereof is administered by subcutaneous injection. Administration by injection, such as by subcutaneous injection, offers the advantage of better comfort for the subject or patient and the opportunity to administer to a subject or patient outside of a hospital setting. In some embodiments, the polypeptide (e.g. lipidated polypeptide) or pharmaceutical composition thereof is administered by self-administration.

In some embodiments the subject or patient is a mammal, in particular a human.

In some embodiments, the polypeptide or pharmaceutical composition is administered to the subject in combination with insulin.

### Articles of Manufacture and Kits

In other aspects, the present invention provides an article of manufacture comprising the polypeptides (e.g. lipidated polypeptides) or pharmaceutical compositions of the invention.

In yet other aspects, the present invention provides a kit comprising the polypeptides (e.g. lipidated polypeptides) or pharmaceutical compositions of the invention. The kit may comprise a package containing the polypeptide (e.g. lipidated polypeptide) or pharmaceutical composition, optionally with instructions. In some embodiments, the polypeptides (e.g. lipidated polypeptides) or pharmaceutical compositions of the invention are formulated in single dose vials or a container closure system (e.g. pre-filled syringe). Optionally associated with such container(s) can be a notice in the form prescribed by a governmental agency regulating the manufacture, use or sale of pharmaceuticals or biological products, which notice reflects approval by the agency of manufacture, use or sale for human administration.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure.

Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise. Thus, for example, reference to “an agent” includes a plurality of such agents and reference to “the agent” includes reference to one or more agents and equivalents thereof known to those skilled in the art, and so forth.

“About” may generally mean an acceptable degree of error for the quantity measured given the nature or precision of the measurements. Exemplary degrees of error are within 20 percent (%), typically, within 10%, and more typically, within 5% of a given value or range of values. Optionally, the term “about” shall be understood herein as plus or minus (±) 5%, optionally ±4%, ±3%, ±2%, ±1%, ±0.5%, ±0.1%, of the numerical value of the number with which it is being used.

Embodiments described herein as “comprising” one or more features may also be considered as disclosure of the corresponding embodiments “consisting of” such features.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In the context of the present disclosure other examples and variations of the polypeptides (e.g. lipidated polypeptides) and methods described herein will be apparent to a person of skill in the art.

Other examples and variations are within the scope of the disclosure, as set out in the appended claims.

All documents cited herein are each entirely incorporated by reference herein, including all data, tables, Figures, and text presented in the cited documents.

### EXAMPLES

### Example 1: Generation of Lipidated Pramlintide Analogue Peptides

Lipidated pramlintide analogue peptides were synthesized as C-terminal carboxamides using rink amide MBHA resin (100-200 mesh). All peptides were prepared by automated synthesis using a Liberty Blue™ microwave solid phase peptide synthesizer (CEM Corporation, NC, USA) using the Fmoc/tBu protocol. Manufacturer-supplied protocols were applied for coupling of amino acids in DMF and deprotection of Fmoc protecting group using piperidine in DMF (20% v/V). Asparagine, cysteine, glutamine and histidine were incorporated as their sidechain trityl (Trt) derivatives. Lysine was incorporated as the sidechain tert-butyloxycarbonyl (Boc) derivative. Serine, threonine and tyrosine were incorporated as sidechain tert-butyl (tBu) ethers, and aspartate and glutamate as their sidechain OtBu esters. Arginine was incorporated as the sidechain 2,2,4,6,7-pentamethyldihydrobenzofuran-5-sulfonyl (Pbf) derivative.

Boc-Lys (Fmoc) was incorporated when a subsequent chemical modification of the N-terminal lysine side chain was required. Upon completion of the peptide chain elongation, coupling of an albumin binding moiety, such as a lipid, was performed manually using HATU as a coupling reagent in the presence of DIPEA.

Peptides were cleaved from the solid support by treatment with a mixture of TFA:TIS:EDT:thioanisole:water (90:2.5:2.5:2.5:2.5 v/v) for 4 h with agitation at room temperature. Thereafter, the cleavage mixtures were filtered, concentrated in vacuo, precipitated and washed with diethyl ether and solids were isolated by centrifugation. The linear crude peptides were dried under a flow of nitrogen and dissolved in 20% MeCN in water (v/v) with 1% TFA (v/V) and filtered. The crude linear peptides were purified using a preparative RP-HPLC on a Varian SD-1 Prep Star binary pump system, monitoring by UV absorption at 210 nm using an Xbridge C18-A stationary phase (19.0×250 mm, 5 micron) column eluting a linear solvent gradient of 25-70% MeCN (0.1% TFA v/v) in water (0.1% TFA v/v) over 25 min.

The linear purified peptides were cyclised by treatment with iodine (1% w/v in methanol) for 10 min at room temperature and excess iodine was reduced by treatment with ascorbic acid (1% w/v in water). The cyclic crude peptides were re-purified as described above. The purified fractions were pooled, frozen and lyophilised.

LC/MS characterisation of purified peptides were performed on a Waters MassLynx 3100 platform using a XBridge C18 stationary phase (4.6×100 mm, 3 micron) eluting a linear binary gradient of 10-90% MeCN (0.1% TFA v/v) in water (0.1% TFA v/v) over 10 minutes at 1.5 mL/min at ambient temperature. Analytes were detected by both UV absorption at 210 nm and ionization using a Waters 3100 mass detector (ESI+ mode). Analytical RP-HPLC characterisation was performed on an Agilent 1260 Infinity system using an Agilent Polaris C8-A stationary phase (4.6×100 mm, 3 micron) eluting a linear binary gradient of 10-90% MeCN (0.1% TFA v/v) in water (0.1% TFA v/v) at 1.5 mL/min over 15 minutes at 40° C.

### Example 2: In Vitro Potency of Lipidated Pramlintide Analogue Peptides in Human or Rat Amylin or Calcitonin Receptor Cells

The functional activities of lipidated pramlintide analogue peptides, such as CAMP production, were tested in 1321N1 cell line with stable recombinant expression of human calcitonin receptor (hCTR) or human amylin receptor (calcitonin receptor co-expressed with receptor activity modifying protein, RAMP3) (hAMYR3) or HEK cells with stable recombinant expression of rat calcitonin receptor (rat CTR) or rat amylin receptor (calcitonin receptor co-expressed with receptor activity modifying protein, RAMP3) (Rat AMYR3).

Cryopreserved cell stock was thawed rapidly in a water-bath, suspended in assay buffer (0.1% BSA (Sigma #A3059) in HBSS (Sigma #H8264) with 25 mM HEPES, pH 7.4 and containing 0.5 mM IB MX (Sigma #17018)) and spun at 240×g for 5 minutes. Cells were re-suspended in assay buffer at a batch-dependent optimized concentration (e.g. hCTR cells at 0.125×10⁵ cells/mL, hAMYR3 cells at 0.125×10⁵ cells/mL, rat CTR cells at at 1×10⁵ cells/mL, rat AMYR3 at 2×10⁵ cells/mL).

The test peptide stock was prepared in DMSO and diluted in assay buffer to reach stated concentrations and transferred in duplicates into a 384-black shallow well microtitre assay plate (Corning #3676). Cells were added to the assay plate, incubated at room temperature for 30 minutes and the CAMP level measured using CAMP dynamic 2 HTRF kit (Cisbio, Cat #62AM4PEJ), following the two step protocol as per manufacturer's recommendations. The plates were read on an Envision (Perkin Elmer) using excitation wavelength of 320 nm and emission wavelengths of 620 nm & 665 nm.

Data was transformed to % Delta F as described in the manufacturer's guidelines and analyzed as percent activation of maximal amylin or calcitonin effect by 4-parameter logistic fit to determine EC50 values. The selectivity of a peptide to hAMYR vs hCTR is defined as a ratio of EC50 values at the two receptors.

All tested compounds show measurable potency in hAMYR and hCTR. Analogues that show >10 fold selectivity for hAMYR over hCTR are preferred.

TABLE 5
in vitro potency of lipidated pramlintide analogues at
human amylin3 and calcitonin receptors

| | EC50 (pM) | | |
|---|---|---|---|
| Peptide | hAMYR3 | hCTR | Ratio |
| 1 | 10 | 160 | 16 |
| 3 | 246 | 1353 | 5 |
| 4 | 248 | 7532 | 30 |
| 5 | 174 | 3605 | 21 |
| 6 | 68 | 310 | 5 |
| 7 | 173 | 2159 | 12 |
| 8 | 375 | 20228 | 54 |
| 9 | 320 | 13521 | 42 |
| 10 | 183 | 2728 | 15 |
| 11 | 177 | 3640 | 21 |
| 12 | 158 | 1224 | 8 |
| 13 | 196 | 2043 | 10 |
| 14 | 220 | 1350 | 6 |
| 15 | 522 | 5461 | 10 |
| 16 | 221 | 2018 | 9 |
| 17 | 365 | 7083 | 19 |
| 18 | 319 | 4686 | 15 |
| 19 | 278 | 4041 | 15 |
| 20 | 174 | 4593 | 26 |
| 21 | 136 | 3408 | 25 |
| 22 | 162 | 6539 | 40 |
| 23 | 238 | 589 | 2 |
| 24 | 127 | 1857 | 15 |
| 25 | 105 | 1248 | 12 |
| 26 | 409 | 2797 | 7 |
| 27 | 134 | 731 | 5 |
| 28 | 390 | 9142 | 23 |
| 29 | 243 | 8211 | 34 |
| 30 | 873 | 10549 | 12 |
| 31 | 349 | 1280 | 4 |
| 32 | 742 | 11149 | 15 |
| 33 | 598 | 15830 | 26 |
| 34 | 877 | 17733 | 20 |
| 35 | 259 | 14153 | 55 |
| 36 | 331 | 6394 | 19 |
| 37 | 249 | 1962 | 8 |
| 38 | 190 | 1049 | 6 |
| 39 | 1048 | 10362 | 10 |
| 40 | 228 | 3329 | 15 |
| 41 | 178 | 1184 | 7 |
| 42 | 470 | 2954 | 6 |
| 43 | 647 | 19046 | 29 |
| 44 | 284 | 6262 | 22 |
| 45 | 588 | 16891 | 29 |
| 46 | 109 | 362 | 3 |
| 47 | 218 | 468 | 2 |
| 48 | 126 | 5273 | 42 |
| 49 | 334 | 4142 | 12 |
| 50 | 349 | 18927 | 54 |
| 51 | 126 | 1645 | 13 |
| 52 | 89 | 1104 | 12 |
| 53 | 98 | 235 | 2 |
| 54 | 7587 | 1507 | 0.2 |
| 55 | 678 | 1874 | 3 |
| 56 | 70 | 493 | 7 |
| 58 | 71 | 65 | 1 |
| 59 | 157 | 809 | 5 |
| 60 | 96 | 1027 | 11 |
| 61 | 99 | 654 | 7 |
| 62 | 167 | 7249 | 43 |
| 63 | 76 | 375 | 5 |
| 64 | 149 | 1278 | 9 |
| 65 | 207 | 6400 | 31 |
| 66 | 418 | 7011 | 17 |
| 67 | 132 | 4006 | 30 |
| 68 | 192 | 1774 | 9 |
| 69 | 164 | 1572 | 10 |
| 70 | 235 | 5405 | 23 |
| 71 | 211 | 1385 | 7 |
| 72 | 227 | 4004 | 18 |
| 73 | 256 | 1821 | 7 |
| 74 | 356 | 4574 | 13 |
| 75 | 161 | 15998 | 99 |
| 76 | 209 | 22291 | 107 |
| 77 | 231 | 2520 | 11 |
| 78 | 288 | 3147 | 11 |
| 79 | 659 | 4799 | 7 |
| 80 | 407 | 2970 | 7 |
| 81 | 404 | 2764 | 7 |
| 82 | 514 | 4277 | 8 |
| 83 | 599 | 6342 | 11 |
| 84 | 118 | 401 | 3 |
| 85 | 806 | 3104 | 4 |
| 86 | 119 | 311 | 3 |
| 87 | 71 | 74 | 1 |
| 88 | 281 | 1172 | 4 |
| 89 | 447 | 8239 | 18 |
| 90 | 1056 | 15595 | 15 |
| 91 | 200 | 2357 | 12 |
| 92 | 341 | 44414 | 130 |
| 94 | 322 | 1903 | 6 |
| 95 | 691 | 1172 | 2 |
| 96 | 556 | 859 | 2 |
| 97 | 684 | 944 | 1 |
| 98 | 942 | 972 | 1 |
| 99 | 3798 | 1825 | 0.5 |
| 100 | 364 | 459 | 1 |
| 101 | 177 | 441 | 2 |
| 102 | 479 | 1630 | 3 |
| 103 | 161 | 1881 | 12 |
| 104 | 79 | 1816 | 23 |
| 106 | 473 | 3420 | 7 |
| 107 | 1071 | 26264 | 25 |
| 108 | 801 | 6808 | 8 |
| 109 | 82 | 176 | 2 |
| 110 | 204 | 541 | 3 |
| 111 | 403 | 1875 | 5 |
| 112 | 861 | 1335 | 2 |
| 113 | 1173 | 18495 | 16 |
| 114 | 1317 | 15796 | 12 |
| 115 | 703 | 7645 | 11 |
| 116 | 1207 | 9557 | 8 |
| 117 | 770 | 3190 | 4 |
| 118 | 417 | 792 | 2 |
| 119 | 1461 | 34657 | 24 |
| 120 | 1769 | 10717 | 6 |
| 121 | 1328 | 6074 | 5 |
| 122 | 655 | 6181 | 9 |
| 123 | 873 | 4793 | 5 |
| 124 | 1129 | 6633 | 6 |
| 125 | 1201 | 10197 | 8 |
| 126 | 445 | 8453 | 19 |
| 127 | 364 | 7127 | 19.6 |
| 128 | 118 | 568 | 4.8 |
| 129 | 553 | 5750 | 10.4 |
| 130 | 446 | 6686 | 15 |
| 131 | 737 | 15143 | 20.5 |
| 132 | 416 | 3848 | 9.3 |
| 133 | 378 | 2578 | 6.8 |
| 134 | 281 | 2223 | 7.9 |
| 135 | 296 | 345 | 1.2 |
| 136 | 122 | 101 | 0.8 |
| 137 | 238 | 1504 | 6.3 |
| 138 | 612 | 2506 | 4.1 |
| 139 | 285 | 1043 | 3.7 |
| 140 | 656 | 6046 | 9.2 |
| 141 | 414 | 3950 | 9.5 |
| 142 | 730 | 2270 | 3.1 |
| 143 | 592 | 6937 | 11.7 |
| 144 | 138 | 725 | 5.3 |
| 145 | 417 | 1876 | 4.5 |
| 146 | 2533 | 4874 | 1.9 |
| 147 | 802 | 1508 | 1.9 |
| 148 | 191 | 728 | 3.8 |
| 149 | 130 | 1200 | 9.2 |
| 150 | 179 | 4181 | 23.4 |
| 151 | 61 | 898 | 14.7 |
| 154 | 574 | 1540 | 2.7 |
| 155 | 329 | 3930 | 11.9 |
| 156 | 3749 | 83771 | 22.3 |

TABLE 6
in vitro potency of lipidated pramlintide analogues
at rat amylin3 and calcitonin receptors

| | EC50 (pM) | |
|---|---|---|
| Peptide | Rat AMYR3 | Rat CTR |
| 1 | 0.4 | 70.0 |
| 3 | 2.9 | 186.0 |
| 8 | 25.6 | 19856.1 |
| 9 | 17.8 | 9724.4 |
| 10 | 12.1 | 2781.5 |
| 12 | 5.0 | 106.8 |
| 15 | 10.3 | 1821.8 |
| 18 | 16.5 | 3086.9 |
| 20 | 10.4 | 1854.2 |
| 24 | 17.7 | 754.9 |
| 35 | 5.9 | 514.1 |
| 38 | 5.1 | 20.3 |
| 40 | 12.0 | 782.1 |
| 41 | 8.2 | 924.8 |
| 43 | 14.5 | 3650.9 |
| 44 | 18.5 | 2237.3 |
| 48 | 4.8 | 2431.0 |
| 112 | 16.0 | 258.5 |
| 113 | 32.6 | 10375.7 |
| 114 | 12.2 | 4873.7 |
| 115 | 22.6 | 22552.0 |
| 116 | 23.7 | 9052.7 |
| 129 | 11.2 | 2869.2 |
| 140 | 19.5 | 1218.4 |
| 149 | 5.6 | 521.8 |

### Example 3: Thioflavin T Fibrillation Assay

Peptide aggregation that form fibrils is an indication of physical instability. Fibril formation in solution poses a significant risk for the stability of injectable peptide drug products. Thioflavin T (ThT) fibrillation assay is a useful tool to assess the aggregation kinetics of a peptide or protein under accelerated and stressed conditions that can be used to forecast the long-term viability of a compound in solution.

ThT can selectively bind amyloid fibrils and the resultant complex emits strong fluorescence signal at 482 nm when excited at 450 nm (Anal Biochem. 1989 March; 177 (2): 244-9). Monitoring of the change in fluorescence signal is an established method to study the fibril forming potential of peptides and proteins.

ThT (purchased from Sigma Aldrich) stock solution is prepared by dissolving the ThT powder in Milli-Q water and filtered to obtain a 0.25 mM solution. The concentration of the solution is measured at 412 nm using an extinction coefficient of 36 mM-1 cm-1. Test peptides were dissolved at 1 mg/mL in 25 mM sodium acetate buffer pH 4.0.

100 μL aliquot of peptide solution and 5 μL aliquot of ThT solution were placed in a clear bottom black fluorescence 96-well plate. 5 replicates of each test samples were placed in the same row of the plate. Buffer was placed in control wells for baseline correction. All empty wells were filled with water to prevent evaporation. The plate was sealed with aluminium seal and placed in fluorescence plate reader and incubated for 6 days at 37° C. with intermittent orbital shaking at 500 to 750 rpm. The fluorescence intensity was measured every 30 min using excitation at 444 nm and emission at 480 nm.

The fibril forming potential of the test peptides was determined by measuring the average time taken to detect an increase in baseline corrected fluorescence intensity. A time >144 h indicates no increase in fluorescence intensity, relative to baseline, during the course of the experiment.

Conjugating pramlintide to a lipid (for example, as in SEQ ID NO. 3, 4, 5, 6, 112, 113, increases the fibril-forming tendency as seen in Table 7.

TABLE 7
Tht fibrillation assay of lipidated pramlintide analogues

| | Time taken to detect increase |
|---|---|
| Peptide | in fluorescence intensity (h) |
| 3 | <5 |
| 4 | <5 |
| 5 | 7 |
| 6 | 15 |
| 8 | 7 |
| 9 | 7 |
| 10 | >144 |
| 11 | >144 |
| 12 | >144 |
| 13 | 25 |
| 15 | >144 |
| 17 | >144 |
| 18 | >144 |
| 19 | >144 |
| 20 | >144 |
| 21 | >144 |
| 24 | >144 |
| 25 | >144 |
| 28 | 45 |
| 33 | >144 |
| 40 | >144 |
| 44 | >144 |
| 48 | >144 |
| 66 | >144 |
| 70 | >144 |
| 80 | >144 |
| 103 | <5 |
| 104 | <5 |
| 112 | <5 |
| 113 | <5 |
| 114 | <5 |
| 115 | <5 |
| 129 | >144 |
| 156 | >144 |

### Example 4: Pharmacokinetic Determination Via IV and SC Administration in Sprague Dawley Rats

The objective of the pharmacokinetic (PK) studies were to determine the plasma pharmacokinetic profile of lipidated pramlintide analogue peptides in fasted male SD rats after single intravenous (IV) and subcutaneous (SC) administration. PK studies were performed to determine the half-life (T_{1/2}) of test peptides. T_{1/2} describes the time taken for the maximum plasma concentration (Cmax) of a test substance to halve its steady-state concentration when in circulation.

Male SD rats were purchased from Si Bei Fu Laboratory Animal Technology Co. Ltd (China). The animals were 6-8 weeks old with body weights of 200-300 g on the dosing date. The animals were housed in a 12-hour light/12-hour dark cycle environment and were fasted overnight before dosing. The body weight of the animals were recorded before dosing, 24 h and 48 h post dosing. Animals had free access to food and drinks, and the food consumption was quantified every day.

Test articles were administered at 20 nmol/kg. Blood samples were collected from each animal via Jugular vein. The sampling timepoints are as below.

Blood Samples per test article;

| Group | Route | Animals | | | Time points |
|---|---|---|---|---|---|
| 1 | IV | 1 | 2 | 3 | 0, 0.033, 0.1, 0.167, 0.25, 0.5, |
| | | | | | 1, 2, 4, 6, 8, 12, 24, 48 h |
| 2 | SC | 4 | 5 | 6 | 0, 0.033, 0.1, 0.167, 0.25, 0.5, |
| | | | | | 1, 2, 4 6, 8, 12, 24, 36, 48 h |

The blood samples were transferred into eppendorf low binding tube containing K2EDTA. Above 0.150 mL blood were collected at each time point. Blood samples were centrifuged at 4,000 g for 5 minutes at 4° C. to obtain plasma. The plasma samples were stored frozen at −75±15° C. until analysis.

Concentrations of the test articles in the plasma samples were analyzed using a LC-MS/MS method. Data acquisition was performed by LabSolution version 5.89 software (Shimadzu, Kyoto, Japan). Data statistics were performed using Excel 97-2003 software. The pharmacokinetic parameters of test articles were calculated using a non-compartmental approach with Phoenix™ WinNonlin®6.1.

The following pharmacokinetic parameters were calculated, whenever possible from the plasma concentration versus time data:
IV administration: T_{1/2}, C₀, AUCₗₐₛₜ, AUC_{inf}, MRT_{inf}, CI, Vss, Number of Points for Regression.
SC administration: Tₘₐₓ, Cₘₐₓ, AUCₗₐₛₜ, AUC_{inf}, MRT_{inf}, F, Number of Points for Regression.

TABLE 8
Half-life of lipidated pramlintide analogues in rats

| | Rat T_{1/2} (h) | |
|---|---|---|
| ID | IV | SC |
| 3 | 14 | — |
| 4 | 12 | 12 |
| 5 | 10.5 | 9.4 |
| 6 | 9.5 | 17.6 |
| 8 | 4.2 | 6.2 |
| 9 | 5.9 | 7.8 |
| 10 | 10.1 | 8.8 |
| 12 | 9.2 | 9.7 |
| 15 | 10.4 | 11.5 |
| 18 | 12 | 13.6 |
| 19 | 6.6 | 9.3 |
| 20 | 10 | 10.8 |
| 21 | 8 | 8.1 |
| 24 | 9.4 | 10.5 |
| 25 | 6.9 | 8 |
| 35 | 5.4 | 8.1 |
| 36 | 7.2 | 11.6 |
| 38 | 6.6 | 9 |
| 40 | 7.4 | 8.1 |
| 41 | 8.3 | 10.1 |
| 43 | 9.3 | 10 |
| 44 | 8.9 | 11 |
| 48 | 5.6 | 8.1 |
| 64 | 6 | 5 |
| 65 | 6 | 9.3 |
| 66 | 9.4 | 12.2 |
| 69 | 6.3 | 8.5 |
| 70 | 10.3 | 12.5 |
| 80 | 9.7 | 9.9 |
| 112 | 11.8 | 19 |
| 113 | 11.2 | 18.2 |
| 114 | 13 | 21.6 |
| 115 | 14.9 | 20.3 |
| 117 | 9.8 | 12.9 |
| 129 | 10.3 | 9 |
| 140 | 10.6 | 12.9 |
| 149 | 6.9 | 9.3 |
| 155 | 7.7 | 8.9 |

Pharmacokinetic studies show that the terminal half-life of amylin in rats is around 13 minutes, and the half-life for pramlintide in human is ˜20-45 minutes (Roth J D et. al. GLP-1R and amylin agonism in metabolic disease: complementary mechanisms and future opportunities. Br J Pharmacol. 2012; 166 (1): 121-136). The lipidated polypeptides show marked improvement in prolonging circulatory T_{1/2} compared to pramlintide.

### Example 6: Rat Acute Food Intake Study

Male Sprague Dawley rats were obtained from Taconic Denmark, ApS at approximately 7 weeks of age. Rats were implanted with a microchip for identification, housed 4/cage with enrichment, free access to food and water, and allowed one week acclimatisation while non-invasive characterization was performed. Rats were on a 12:12 light:dark cycle that switches at 1 pm: 1 am. Food intake was monitored via the HM2 system (Lafayette Instrument) that allows for monitoring in a home cage. As each rat enters an access tunnel to feed, an IR beam is broken, and the implanted microchip is read. Resulting changes to food weight is then assigned to the specific animal. Social order has shown no impact to overall feeding patterns and amounts.

Rats were sorted into groups based on Day-1 body weight and 24-hour accumulated food intake (n=7 per group). On Day 0 rats were weighed, then fasted for 6 hours. Thirty (30) minutes prior to the reintroduction of food, rats were dosed subcutaneously (5 ml/kg) with 20 nmol/kg of test compound or 60 nmol/kg peptide 1 (pramlintide) diluted in an appropriate vehicle, after which food was returned, and lights went out. Automated food intake was monitored for the following 3 days, and rats were weighed once per day.

Food intake per rat was batched into 1-hour intervals and integrated into Gubra's GubraView data management system. Discrete food intake data was exported into MS Excel from which cumulative food intake data was generated. Cumulative food intake data was then transposed into GraphPad Prism (v8.0.1) for analysis of dark period feeding

The lipidated polypeptides show marked suppression of food intake compared to pramlintide.

TABLE 9
Effect of lipidated pramlintide analogues on food uptake in lean rats

| | Cummulative food intake | | |
|---|---|---|---|
| | (% vehicle treated intake)) | | |
| Peptide | At 12 h | At 24 h | At 48 h |
| 1 | 71.6 | 85.6 | 98.7 |
| 3 | 13.4 | 14.4 | 30.3 |
| 8 | 28.1 | 42.7 | 68.8 |
| 9 | 21.9 | 30.8 | 56.5 |
| 10 | 18.8 | 24.6 | 46.6 |
| 12 | 9.4 | 7.5 | 23.6 |
| 15 | 45.2 | 49.1 | 51 |
| 18 | 45.8 | 46.9 | 68.8 |
| 20 | 27 | 26.2 | 49.7 |
| 24 | 18.7 | 19.4 | 40.6 |
| 35 | 49.8 | 44.8 | 72 |
| 38 | 6.9 | 5.5 | 13.7 |
| 40 | 60.7 | 51.8 | 72.4 |
| 41 | 44.6 | 38.5 | 58.8 |
| 43 | 60.8 | 55.6 | 46.6 |
| 44 | 33.5 | 39.2 | 60.7 |
| 48 | 27.5 | 32.6 | 57.1 |
| 112 | 15.8 | 15.9 | 22.1 |
| 113 | 69.5 | 65.8 | 77.9 |
| 114 | 42.3 | 45.1 | 53.8 |
| 115 | 42.4 | 41.3 | 52.2 |
| 116 | 51.7 | 43.1 | 50.2 |
| 129 | 29 | 32.4 | 51 |
| 140 | 62.4 | 54.4 | 56.5 |
| 149 | 31 | 31.8 | 17.5 |

1-93. (canceled)

94. A method of treating a disease or disorder in a subject, the method comprising administering a polypeptide or a pharmaceutically acceptable salt thereof to the subject, wherein the polypeptide comprises the amino acid sequence selected from the group consisting of:
K(γE-C18diacid)K[CNTATC]ATQRLANFLRHSSNN(αMePhe) GPILPPTEVGSNTY-amide (SEQ ID NO: 15),
K(γE-C18diacid)[CNTATC]ATQRLANFLRHSSNN(αMePhe) GPILPPTEVGSNTY-amide (SEQ ID NO: 16),
K(O2Oc-O2Oc-γE-C18diacid)[CNTATC]ATQRLANFLRHSSNN(αMePhe) GPILPPTEVGSNTY-amide (SEQ ID NO: 17), and
K(γE-γE-C18diacid)[CNTATC]ATQRLANFLRHSSNN(αMePhe)GPILPPTEVGSNTY-amide (SEQ ID NO: 18).

95. The method according to claim 94, wherein the disease or disorder is obesity, metabolic disease, an obesity-related condition, eating disorder, Alzheimer's disease, hepatic steatosis (“fatty liver”), kidney failure, arteriosclerosis (e.g. atherosclerosis), cardiovascular disease, macrovascular disease, microvascular disease, diabetic heart (including diabetic cardiomyopathy and heart failure as a diabetic complication), coronary heart disease, peripheral artery disease or stroke, cancer, dumping syndrome, hypertension e.g. pulmonary hypertension, or dyslipidemia e.g. atherogenic dyslipidemia, cholescystitis, or short bowel syndrome.

96. The method according to claim 95, wherein the obesity-related condition is overweight, morbid obesity, obesity prior to surgery, obesity-linked inflammation, obesity-linked gallbladder disease, sleep apnea and respiratory problems, hyperlipidemia, degeneration of cartilage, osteoarthritis, or reproductive health complications of obesity or overweight such as infertility.

97. The method according to claim 95, wherein the metabolic disease is diabetes, type 1 diabetes, type 2 diabetes, gestational diabetes, pre-diabetes, insulin resistance, impaired glucose tolerance (IGI), disease states associated with elevated blood glucose levels, metabolic disease including metabolic syndrome, or hyperglycemia e.g. abnormal postprandial hyperglycemia.

98. The method according to claim 94, wherein the polypeptide comprises the amino acid sequence
K(γEC18diacid)K[CNTATC]ATQRLANFLRHSSNN(αMePhe) GPILPPTEVGSNTY-amide (SEQ ID NO: 15).

99. The method according to claim 94, wherein the polypeptide comprises the amino acid sequence K(γEC18diacid)[CNTATC]ATQRLANFLRHSSNN(αMePhe) GPILPPTEVGSNTY-amide (SEQ ID NO: 16).

100. The method according to claim 94, wherein the polypeptide comprises the amino acid sequence
K(O2Oc-O2OcγEC18diacid)[CNTATC]ATQRLANFLRHSSNN(αMePhe) GPILPPTEVGSNTY-amide (SEQ ID NO: 17).

101. The method according to claim 94, wherein the polypeptide comprises the amino acid sequence K(γE-γE-C18diacid)[CNTATC]ATQRLANFLRHSSNN(αMePhe) GPILPPTEVGSNTY-amide (SEQ ID NO: 18).

102. A method of inhibiting or reducing weight gain, promoting weight loss, reducing food intake, and/or reducing excess body weight in a subject, the method comprising administering a polypeptide or a pharmaceutically acceptable salt thereof to the subject, wherein the polypeptide comprises the amino acid sequence selected from the group consisting of:
K(γE-C18diacid)K[CNTATC]ATQRLANFLRHSSNN(@MePhe) GPILPPTEVGSNTY-amide (SEQ ID NO: 15),
K(γE-C18diacid)[CNTATC]ATQRLANFLRHSSNN(αMePhe) GPILPPTEVGSNTY-amide (SEQ ID NO: 16),
K(O2Oc-O2Oc-γE-C18diacid)[CNTATC]ATQRLANFLRHSSNN(αMePhe) GPILPPTEVGSNTY-amide (SEQ ID NO: 17), and
K(γE-γE-C18diacid)[CNTATC]ATQRLANFLRHSSNN(αMePhe) GPILPPTEVGSNTY-amide (SEQ ID NO: 18).

103. The method according to claim 102, wherein the polypeptide comprises the amino acid sequence
K(γE-C18diacid)K[CNTATC]ATQRLANFLRHSSNN(αMePhe) GPILPPTEVGSNTY-amide (SEQ ID NO: 15).

104. The method according to claim 102, wherein the polypeptide comprises the amino acid sequence
K(γE-C18diacid)[CNTATC]ATQRLANFLRHSSNN(αMePhe) GPILPPTEVGSNTY-amide (SEQ ID NO: 16).

105. The method according to claim 102, wherein the polypeptide comprises the amino acid sequence
K(O2Oc-O2OcγEC18diacid)[CNTATC]ATQRLANFLRHSSNN(αMePhe) GPILPPTEVGSNTY-amide (SEQ ID NO: 17).

106. The method according to claim 102, wherein the polypeptide comprises the amino acid sequence
K(γE-γE-C18diacid)[CNTATC]ATQRLANFLRHSSNN(@MePhe) GPILPPTEVGSNTY-amide (SEQ ID NO: 18).

107. The method according to claim 94, wherein the polypeptide, the pharmaceutically acceptable salt or the pharmaceutical composition is administered to the subject by subcutaneous injection.

108. The method according to claim 102, wherein the polypeptide, the pharmaceutically acceptable salt or the pharmaceutical composition is administered to the subject by subcutaneous injection.

109. The method according to claim 94, wherein the polypeptide, the pharmaceutically acceptable salt or the pharmaceutical composition is administered to the subject by self-administration.

110. The method according to claim 102, wherein the polypeptide, the pharmaceutically acceptable salt or the pharmaceutical composition is administered to the subject by self-administration.


SEQUENCE LISTING (311 sequences)

SEQ ID NO: 1 - protein, 37 aa, synthetic construct
  REGION 1..37: note = Description of Artificial Sequence: Synthetic polypeptide
  SITE 2..7: note = Disulphide bridge between Cys residues
KCNTATCATQRLANFLVHSSNNFGPILPPTNVGSNTY

SEQ ID NO: 2 - protein, 41 aa, synthetic construct
  REGION 1..41: note = Description of Artificial Sequence: Synthetic polypeptide
  VARIANT 1: note = Lys (albumin binding moiety), or absent
  VARIANT 2: note = Gly or absent
  VARIANT 3: note = Gly or absent
  VARIANT 4: note = Gly, Lys (albumin binding moiety), or absent
  VARIANT 5: note = Lys, Lys (albumin binding moiety), or absent
  VARIANT 8: note = Thr, Ile, Ala
  VARIANT 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid
  SITE 1..41: note = Variant residues given in the sequence have no preference with respect to those in the annotations for variant positions
XXXXXCNXATCATQRLAXXXXHSXXXXXXXXXXTXXXXXXX

SEQ ID NO: 3 - protein, 38 aa, synthetic construct
  REGION 1..38: note = Description of Artificial Sequence: Synthetic polypeptide
  MOD_RES 1: note = C18diacid-gamma-Glu
  SITE 3..8: note = Disulphide bridge between Cys residues
XKCNTATCATQRLANFLVHSSNNFGPILPPTNVGSNTY

SEQ ID NO: 4 - protein, 42 aa, synthetic construct
  REGION 1..42: note = Description of Artificial Sequence: Synthetic polypeptide
  MOD_RES 1: note = C18diacid-gamma-Glu
  MOD_RES 2: note = gamma-Glu
  SITE 7..12: note = Disulphide bridge between Cys residues
XXGGGKCNTATCATQRLANFLVHSSNNFGPILPPTNVGSNTY

SEQ ID NO: 5 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
XCNTATCATQRLANFLVHSSNNFGPILPPTNVGSNTY

SEQ ID NO: 6 - protein, 37 aa, synthetic construct; identical to SEQ ID NO: 5
  REGION 1..37: note = Description of Artificial Sequence: Synthetic polypeptide
  MOD_RES 1: note = Lys, wherein the side chain of Lys is connected to O2Oc-O2Oc-gamma-Glu-C18diacid
  SITE 2..7: note = Disulphide bridge between Cys residues

SEQ ID NO: 7 - protein, 41 aa, synthetic construct
  REGION 1..41: note = Description of Artificial Sequence: Synthetic polypeptide
  MOD_RES 1: note = Lys, wherein the side chain of Lys is connected to O2Oc-O2Oc-gamma-Glu-C18diacid
  SITE 6..11: note = Disulphide bridge between Cys residues
XGGGKCNTATCATQRLANFLVHSSNNFGPILPPTNVGSNTY

SEQ ID NO: 8 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2,4-diaminobutanoic acid
XCNTATCATQRLANFLVHSSXNFPAILSPTNVGSNTY

SEQ ID NO: 9 - protein, 37 aa, synthetic construct; identical to SEQ ID NO: 8
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 10 - protein, 37 aa, synthetic construct
  REGION 1..37: note = Description of Artificial Sequence: Synthetic polypeptide
  MOD_RES 1: note = C18diacid-gamma-Glu
  SITE 2..7: note = Disulphide bridge between Cys residues
  MOD_RES 23: note = (S)-2-amino-2-methyl-3-phenylpropanoic acid
XCNTATCATQRLAEFLRHSSNNXGPILPPTNVGSNTY

SEQ ID NO: 11 - protein, 37 aa, synthetic construct; identical to SEQ ID NO: 10
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid

SEQ ID NO: 12 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 24: note = (S)-2-amino-2-methyl-3-phenylpropanoic acid
XKCNTATCATQRLAEFLRHSSNNXGPILPPTNVGSNTY

SEQ ID NO: 13 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLVHSSNNXGPILPPTNVGSNTY

SEQ ID NO: 14 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 24: note = (S)-2-amino-2-methyl-3-phenylpropanoic acid
XKCNTATCATQRLANFLVHSSNNXGPILPPTNVGSNTY

SEQ ID NO: 15 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 24: note = (S)-2-amino-2-methyl-3-phenylpropanoic acid
XKCNTATCATQRLANFLRHSSNNXGPILPPTEVGSNTY

SEQ ID NO: 16 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLRHSSNNXGPILPPTEVGSNTY

SEQ ID NO: 17 - protein, 37 aa, synthetic construct; identical to SEQ ID NO: 16
  REGION 1..37: note = Description of Artificial Sequence: Synthetic polypeptide
  MOD_RES 1: note = Lys, wherein the side chain of Lys is connected to O2Oc-O2Oc-gamma-Glu-C18diacid
  SITE 2..7: note = Disulphide bridge between Cys residues
  MOD_RES 23: note = (S)-2-amino-2-methyl-3-phenylpropanoic acid

SEQ ID NO: 18 - protein, 37 aa, synthetic construct; identical to SEQ ID NO: 16
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid

SEQ ID NO: 19 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLRHSSNNXGPILPPTNVGSNTY

SEQ ID NO: 20 - protein, 37 aa, synthetic construct; identical to SEQ ID NO: 19
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid

SEQ ID NO: 21 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLVHSSNNXGPILPPTNVGSRTY

SEQ ID NO: 22 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 20: note = (S)-2-amino-3-hydroxy-2-methylpropanoic acid
XCNTATCATQRLANFLVHSXNNFGPILPPTNVGSNTY

SEQ ID NO: 23 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLVHSSNNXGPILPPTEVGSNTY

SEQ ID NO: 24 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 23: note = (S)-2-amino-2-methyl-3-phenylpropanoic acid
XCNTATCATQRLANFLVHSSNNXGPILPPTRVGSNTY

SEQ ID NO: 25 - protein, 37 aa, synthetic construct; identical to SEQ ID NO: 24
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 23: note = (S)-2-amino-2-methyl-3-phenylpropanoic acid

SEQ ID NO: 26 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 17: note = 2-amino-2-methylpropanoic acid
  MOD_RES 23: note = (S)-2-amino-2-methyl-3-phenylpropanoic acid
XCNTATCATQRLANFLXHSSNNXGPILPPTNVGSNTY

SEQ ID NO: 27 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2,4-diaminobutanoic acid
XCNIATCATQRLANFLVHSSXNFGPILPPTNVGSRTY

SEQ ID NO: 28 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2,4-diaminobutanoic acid
XKCNTATCATQRLANFLVHSSXNFGPILPPTEVGSNTY

SEQ ID NO: 29 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2,4-diaminobutanoic acid
XKCNTATCATQRLANFLVHSSXNFGPILPPTNVGSNTY

SEQ ID NO: 30 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2,4-diaminobutanoic acid
XKCNTATCATQRLANFLXHSSXNFGPILPPTEVGSNTY

SEQ ID NO: 31 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2,4-diaminobutanoic acid
  MOD_RES 26: note = 2-amino-2-methylpropanoic acid
XKCNTATCATQRLANFLVHSSXNFGXILPPTNVGSNTY

SEQ ID NO: 32 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 15: note = 2,4-diaminobutanoic acid
  MOD_RES 24: note = (S)-2-amino-2-methyl-3-phenylpropanoic acid
XKCNTATCATQRLAXFLVHSSNNXGPILPPTEVGSNTY

SEQ ID NO: 33 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 15: note = 2,4-diaminobutanoic acid
XKCNTATCATQRLAXFLVHSSNNFGPILPPTNVGSNTY

SEQ ID NO: 34 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 15: note = 2,4-diaminobutanoic acid
XKCNTATCATQRLAXFLVHSSNNFGPILPPTEVGSNTY

SEQ ID NO: 35 - protein, 38 aa, synthetic construct; identical to SEQ ID NO: 29
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid

SEQ ID NO: 36 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2-amino-2-methylpropanoic acid
XKCNTATCATQRLANFLXHSSXNFGPILPPTNVGSNTY

SEQ ID NO: 37 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTNVGSNTY

SEQ ID NO: 38 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLAEFLVHSSXNFGPILPPTNVGSNTY

SEQ ID NO: 39 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLEHSSXNFGPILPPTNVGSNTY

SEQ ID NO: 40 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLVHSSXNFGPILPPTHVGSNTY

SEQ ID NO: 41 - protein, 38 aa, synthetic construct; identical to SEQ ID NO: 28
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid

SEQ ID NO: 42 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLVHSSXNFGPILPPTNVGSETY

SEQ ID NO: 43 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLRHSSXNFGPILPPTEVGSNTY

SEQ ID NO: 44 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLRHSSXNFGPILPPTEVGSNTY

SEQ ID NO: 45 - protein, 37 aa, synthetic construct; identical to SEQ ID NO: 44
  REGION 1..37: note = Description of Artificial Sequence: Synthetic polypeptide
  MOD_RES 1: note = Lys, wherein the side chain of Lys is connected to O2Oc-O2Oc-gamma-Glu-C18diacid
  SITE 2..7: note = Disulphide bridge between Cys residues
  MOD_RES 21: note = 2-amino-2-methylpropanoic acid

SEQ ID NO: 46 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLAHFLVHSSXNFGPILPPTNVGSNTY

SEQ ID NO: 47 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLAHFLVHSSXNFGPILPPTNVGSETY

SEQ ID NO: 48 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLRHSSXNFGPILPPTNVGSNTY

SEQ ID NO: 49 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
XCNTATCATQRLANFLVHSSXNFGPILPPTEVGSNTY

SEQ ID NO: 50 - protein, 37 aa, synthetic construct; identical to SEQ ID NO: 44
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 51 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTHVGSNTY

SEQ ID NO: 52 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTRVGSNTY

SEQ ID NO: 53 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTPVGSNTY

SEQ ID NO: 54 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTNVPSNTY

SEQ ID NO: 55 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLSHSSXNFGPILPPTNVGSPTY

SEQ ID NO: 56 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLVHSSXNFGPILPPTNVGSNTY

SEQ ID NO: 57 - protein, 38 aa, synthetic construct; identical to SEQ ID NO: 29
  REGION 1..38: note = Description of Artificial Sequence: Synthetic polypeptide
  MOD_RES 1: note = Lys, wherein the side chain of Lys is connected to gamma-Glu-gamma-Glu-C18diacid
  SITE 3..8: note = Disulphide bridge between Cys residues
  MOD_RES 22: note = 2-amino-2-methylpropanoic acid

SEQ ID NO: 58 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLVHSSXNFGPILPPTNVGSNTP

SEQ ID NO: 59 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
  MOD_RES 27: note = D-Leu
XCNTATCATQRLANFLVHSSXNFGPIXPPTNVGSNTY

SEQ ID NO: 60 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
  MOD_RES 28: note = D-Pro
XCNTATCATQRLANFLVHSSXNFGPILXPTNVGSNTY

SEQ ID NO: 61 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
  MOD_RES 26: note = D-Ile
XCNTATCATQRLANFLVHSSXNFGPXLPPTNVGSNTY

SEQ ID NO: 62 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 16: note = D-Leu
  MOD_RES 21: note = 2-amino-2-methylpropanoic acid
XCNTATCATQRLANFXVHSSXNFGPILPPTNVGSNTY

SEQ ID NO: 63 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLVHSSXNFGPILPPTRVGSNTY

SEQ ID NO: 64 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLVHSSXNFGPILPPTNVGSRTY

SEQ ID NO: 65 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLRHSSXNFGPILPPTNVGSNTY

SEQ ID NO: 66 - protein, 38 aa, synthetic construct; identical to SEQ ID NO: 65
  REGION 1..38: note = Description of Artificial Sequence: Synthetic polypeptide
  MOD_RES 1: note = Lys, wherein the side chain of Lys is connected to gamma-Glu-gamma-Glu-C18diacid
  SITE 3..8: note = Disulphide bridge between Cys residues
  MOD_RES 22: note = 2-amino-2-methylpropanoic acid

SEQ ID NO: 67 - protein, 37 aa, synthetic construct; identical to SEQ ID NO: 63
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 68 - protein, 37 aa, synthetic construct; identical to SEQ ID NO: 63
  REGION 1..37: note = Description of Artificial Sequence: Synthetic polypeptide
  MOD_RES 1: note = C18diacid-gamma-Glu
  SITE 2..7: note = Disulphide bridge between Cys residues
  MOD_RES 21: note = 2-amino-2-methylpropanoic acid

SEQ ID NO: 69 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLVHSSXNFGPILPPTRVGSNTY

SEQ ID NO: 70 - protein, 38 aa, synthetic construct; identical to SEQ ID NO: 69
  REGION 1..38: note = Description of Artificial Sequence: Synthetic polypeptide
  MOD_RES 1: note = Lys, wherein the side chain of Lys is connected to gamma-Glu-gamma-Glu-C18diacid
  SITE 3..8: note = Disulphide bridge between Cys residues
  MOD_RES 22: note = 2-amino-2-methylpropanoic acid

SEQ ID NO: 71 - protein, 38 aa, synthetic construct; identical to SEQ ID NO: 69
  REGION 1..38: note = Description of Artificial Sequence: Synthetic polypeptide
  MOD_RES 1: note = C18diacid-gamma-Glu
  SITE 3..8: note = Disulphide bridge between Cys residues
  MOD_RES 22: note = 2-amino-2-methylpropanoic acid

SEQ ID NO: 72 - protein, 37 aa, synthetic construct; identical to SEQ ID NO: 64
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 73 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
XKCNTATCATQRLANFLVHSSXNFGPILPPTNVGSRTY

SEQ ID NO: 74 - protein, 38 aa, synthetic construct; identical to SEQ ID NO: 73
  REGION 1..38: note = Description of Artificial Sequence: Synthetic polypeptide
  MOD_RES 1: note = Lys, wherein the side chain of Lys is connected to gamma-Glu-gamma-Glu-C18diacid
  SITE 3..8: note = Disulphide bridge between Cys residues
  MOD_RES 22: note = 2-amino-2-methylpropanoic acid

SEQ ID NO: 75 - protein, 37 aa, synthetic construct; identical to SEQ ID NO: 48
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 76 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 21: note = 2-amino-2-methylpropanoic acid
XCNTATCATQRLANFLRHSSXNFGPILPPTRVGSNTY

SEQ ID NO: 77 - protein, 37 aa, synthetic construct; identical to SEQ ID NO: 76
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid

SEQ ID NO: 78 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 27: note = 2-amino-2-methylpropanoic acid
XKCNTATCATQRLANFLVHSSXNFGPXLPPTNVGSNTY

SEQ ID NO: 79 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 28: note = 2-amino-2-methylpropanoic acid
XKCNTATCATQRLANFLVHSSXNFGPIXPPTNVGSNTY

SEQ ID NO: 80 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 32: note = 2-amino-2-methylpropanoic acid
XKCNTATCATQRLANFLVHSSXNFGPILPPTXVGSNTY

SEQ ID NO: 81 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 34: note = 2-amino-2-methylpropanoic acid
XKCNTATCATQRLANFLVHSSXNFGPILPPTNVXSNTY

SEQ ID NO: 82 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 36: note = 2-amino-2-methylpropanoic acid
XKCNTATCATQRLANFLVHSSXNFGPILPPTNVGSXTY

SEQ ID NO: 83 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 37: note = 2-amino-2-methylpropanoic acid
XKCNTATCATQRLANFLVHSSXNFGPILPPTNVGSNXY

SEQ ID NO: 84 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 35: note = 2-amino-2-methylpropanoic acid
XKCNTATCATQRLANFLVHSSXNFGPILPPTNVGXNTY

SEQ ID NO: 85 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 38: note = 2-amino-2-methylpropanoic acid
XKCNTATCATQRLANFLVHSSXNFGPILPPTNVGSNTX

SEQ ID NO: 86 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2-amino-2-methylpropanoic acid
XKCNTATCATQRLAHFLXHSSXNFGPILPPTEVGSNTY

SEQ ID NO: 87 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2-amino-2-methylpropanoic acid
XKCNTATCATQRLANFLXHSSXNFGPILPPTNVGSNTP

SEQ ID NO: 88 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2-amino-2-methylpropanoic acid
  MOD_RES 29: note = 2-amino-2-methylpropanoic acid
XKCNTATCATQRLANFLVHSSXNFGPILXPTNVGSNTY

SEQ ID NO: 89 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 31: note = 2-amino-2-methylpropanoic acid
XCNTATCATQRLANFLVHSSXNFGPILPPTXVGSNTY

SEQ ID NO: 90 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 31: note = 2-amino-2-methylpropanoic acid
XCNTATCATQRLANFLRHSSXNFGPILPPTXVGSNTY

SEQ ID NO: 91 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 35: note = 2-amino-2-methylpropanoic acid
XCNTATCATQRLANFLVHSSXNFGPILPPTNVGSXTY

SEQ ID NO: 92 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 35: note = 2-amino-2-methylpropanoic acid
XCNTATCATQRLANFLRHSSXNFGPILPPTNVGSXTY

SEQ ID NO: 93 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2,4-diaminobutanoic acid
  MOD_RES 26: note = 2-amino-2-methylpropanoic acid
XKCNTATCATQRLANFLXHSSXNFGXILPPTNVGSNTY

SEQ ID NO: 94 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 27: note = 2-amino-2-methylpropanoic acid
XKCNTATCATQRLANFLXHSSNNFGPXLPPTNVGSNTY

SEQ ID NO: 95 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 28: note = 2-amino-2-methylpropanoic acid
XKCNTATCATQRLANFLXHSSNNFGPIXPPTNVGSNTY

SEQ ID NO: 96 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 29: note = 2-amino-2-methylpropanoic acid
XKCNTATCATQRLANFLXHSSNNFGPILXPTNVGSNTY

SEQ ID NO: 97 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 30: note = 2-amino-2-methylpropanoic acid
XKCNTATCATQRLANFLXHSSNNFGPILPXTNVGSNTY

SEQ ID NO: 98 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 32: note = 2-amino-2-methylpropanoic acid
XKCNTATCATQRLANFLXHSSNNFGPILPPTXVGSNTY

SEQ ID NO: 99 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 33: note = 2-amino-2-methylpropanoic acid
XKCNTATCATQRLANFLXHSSNNFGPILPPTNXGSNTY

SEQ ID NO: 100 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 34: note = 2-amino-2-methylpropanoic acid
XKCNTATCATQRLANFLXHSSNNFGPILPPTNVXSNTY

SEQ ID NO: 101 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 35: note = 2-amino-2-methylpropanoic acid
XKCNTATCATQRLANFLXHSSNNFGPILPPTNVGXNTY

SEQ ID NO: 102 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 36: note = 2-amino-2-methylpropanoic acid
XKCNTATCATQRLANFLXHSSNNFGPILPPTNVGSXTY

SEQ ID NO: 103 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 27: note = D-Leu
XCNTATCATQRLANFLVHSSNNFGPIXPPTNVGSNTY

SEQ ID NO: 104 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 28: note = D-Pro
XCNTATCATQRLANFLVHSSNNFGPILXPTNVGSNTY

SEQ ID NO: 105 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 22: note = 2,4-diaminobutanoic acid
  MOD_RES 26: note = 2-amino-2-methylpropanoic acid
XKCNTATCATQRLANFLSHSSXNFGXILPPTNVGSNTY

SEQ ID NO: 106 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 27: note = 2-amino-2-methylpropanoic acid
XKCNTATCATQRLANFLVHSSNNFGPXLPPTNVGSNTY

SEQ ID NO: 107 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 27: note = 2-amino-2-methylpropanoic acid
XKCNTATCATQRLANFLRHSSNNFGPXLPPTNVGSNTY

SEQ ID NO: 108 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 28: note = 2-amino-2-methylpropanoic acid
XKCNTATCATQRLANFLVHSSNNFGPIXPPTNVGSNTY

SEQ ID NO: 109 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 23: note = 2-amino-2-methylpropanoic acid
XKCNTATCATQRLANFLVHSSNXFGPILPPTNVGSNTY

SEQ ID NO: 110 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 25: note = 2-amino-2-methylpropanoic acid
XKCNTATCATQRLANFLVHSSNNFXPILPPTNVGSNTY

SEQ ID NO: 111 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
XKCNTATCATQRLANFLVHSSNHFGPILPPTNVGSETY

SEQ ID NO: 112 - protein, 38 aa, synthetic construct; identical to SEQ ID NO: 3
  REGION 1..38: note = Description of Artificial Sequence: Synthetic polypeptide
  MOD_RES 1: note = C20diacid-gamma-Glu
  SITE 3..8: note = Disulphide bridge between Cys residues

SEQ ID NO: 113 - protein, 38 aa, synthetic construct
  REGION 1..38: note = Description of Artificial Sequence: Synthetic polypeptide
  MOD_RES 1: note = C20diacid-gamma-Glu
  MOD_RES 2: note = O2Oc-O2Oc-Lys
  SITE 3..8: note = Disulphide bridge between Cys residues
XXCNTATCATQRLANFLVHSSNNFGPILPPTNVGSNTY

SEQ ID NO: 114 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNTATCATQRLANFLVHSSNNFGPILPPTNVGSRTY

SEQ ID NO: 115 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNTATCATQRLANFLVHSSPNFPAILSPTNVGSNTY

SEQ ID NO: 116 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNTATCATQRLAEFLRHSSNNFGPILPPTNVGSNTY

SEQ ID NO: 117 - protein, 37 aa, synthetic construct; identical to SEQ ID NO: 8
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
  MOD_RES 21: note = 2-amino-2-methylpropanoic acid

SEQ ID NO: 118 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNIATCATQRLANFLVHSIANFGPILPPTNVGSRTY

SEQ ID NO: 119 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNTATCATQRLANFLVHSPPNFPAILSPTNVGSNTY

SEQ ID NO: 120 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
XCNAATCATQRLANWLVHSSPNFPAILSPTNVGSNTY

SEQ ID NO: 121 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
  MOD_RES 21: note = 2-amino-2-methylpropanoic acid
  MOD_RES 24: note = (2S,4R)-4-hydroxypyrrolidine-2-carboxylic acid
XCNTATCATQRLANFLVHSSXNFXAILSPTNVGSNTY

SEQ ID NO: 122 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 31: note = 2,4-diaminobutanoic acid
XCNTATCATQRLANFLVHSSXNFPAILSPTXVGSNTY

SEQ ID NO: 123 - protein, 37 aa, synthetic construct; identical to SEQ ID NO: 122
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
  MOD_RES 21: note = 2-amino-2-methylpropanoic acid
  MOD_RES 31: note = 2,4-diaminobutanoic acid

SEQ ID NO: 124 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 21: note = 2-amino-2-methylpropanoic acid
  MOD_RES 35: note = 2,4-diaminobutanoic acid
XCNTATCATQRLANFLVHSSXNFPAILSPTNVGSXTY

SEQ ID NO: 125 - protein, 37 aa, synthetic construct; identical to SEQ ID NO: 124
  REGION 1..37: note = Description of Artificial Sequence: Synthetic polypeptide
  MOD_RES 1: note = Lys, wherein the side chain of Lys is connected to gamma-Glu-gamma-Glu-C20diacid
  SITE 2..7: note = Disulphide bridge between Cys residues
  MOD_RES 21: note = 2-amino-2-methylpropanoic acid
  MOD_RES 35: note = 2,4-diaminobutanoic acid

SEQ ID NO: 126 - protein, 38 aa, synthetic construct; identical to SEQ ID NO: 36
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
  MOD_RES 22: note = 2,4-diaminobutanoic acid

SEQ ID NO: 127 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-gamma-Glu-C18diacid
  SITE 2..7: note = Disulphide bridge between Cys residues
  MOD_RES 17: note = 2-amino-2-methylpropanoic acid
XCNTATCATQRLANFLXHSSNNFGPILPPTNVGSNTY

SEQ ID NO: 128 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLAEFLXHSSNNFGPILPPTNVGSNTY

SEQ ID NO: 129 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGSNTY

SEQ ID NO: 130 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLAEFLXHSSHNFGPILPPTNVGSNTY

SEQ ID NO: 131 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSHNFGPILPPTNVGSNTY

SEQ ID NO: 132 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSPNFGPILPPTNVGSNTY

SEQ ID NO: 133 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSSNFGPILPPTNVGSNTY

SEQ ID NO: 134 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTPVGSNTY

SEQ ID NO: 135 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNHFGPILPPTNVGSNTY

SEQ ID NO: 136 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGSNTP

SEQ ID NO: 137 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSRNFGPILPPTNVGSNTY

SEQ ID NO: 138 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSPNFGPILPPTEVGSNTY

SEQ ID NO: 139 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSPNFGPILPPTNVGSETY

SEQ ID NO: 140 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTEVGSNTY

SEQ ID NO: 141 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGSRTY

SEQ ID NO: 142 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGSETY

SEQ ID NO: 143 - protein, 40 aa, synthetic construct
  REGION 1..40: note = Description of Artificial Sequence: Synthetic polypeptide
  MOD_RES 1: note = Lys, wherein the side chain of Lys is connected to gamma-Glu-C18diacid
  SITE 5..10: note = Disulphide bridge between Cys residues
  MOD_RES 20: note = 2-amino-2-methylpropanoic acid
XGGKCNTATCATQRLANFLXHSSNNFGPILPPTNVGSNTY

SEQ ID NO: 144 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 17: note = 2-amino-2-methylpropanoic acid
XCNTATCATQRLAHFLXHSSNNFGPILPPTNVGSNTY

SEQ ID NO: 145 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGHNTY

SEQ ID NO: 146 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTHVGSETY

SEQ ID NO: 147 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNHFGPILPPTNVGSETY

SEQ ID NO: 148 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPILPPTNVGPNTY

SEQ ID NO: 149 - protein, 37 aa, synthetic construct; identical to SEQ ID NO: 127
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 17: note = 2-amino-2-methylpropanoic acid

SEQ ID NO: 150 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 17: note = 2-amino-2-methylpropanoic acid
  MOD_RES 21: note = 2,4-diaminobutanoic acid
XCNTATCATQRLANFLXHSSXNFGPILPPTNVGSNTY

SEQ ID NO: 151 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 17: note = 2-amino-2-methylpropanoic acid
  MOD_RES 27: note = D-Leu
XCNTATCATQRLANFLXHSSNNFGPIXPPTNVGSNTY

SEQ ID NO: 152 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSNNFGPRLPPTNVGSNTY

SEQ ID NO: 153 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSKNFGPILPPTNVGSNTY

SEQ ID NO: 154 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 18: note = 2-amino-2-methylpropanoic acid
XKCNTATCATQRLANFLXHSSGNFGPILPPTNVGSNTY

SEQ ID NO: 155 - protein, 37 aa, synthetic construct
  REGION 1..37: note = Description of Artificial Sequence: Synthetic polypeptide
  MOD_RES 1: note = Lys, wherein the side chain of Lys is connected to gamma-Glu-C18diacid
  SITE 2..7: note = Disulphide bridge between Cys residues
  MOD_RES 17: note = 2-amino-2-methylpropanoic acid
XCNTATCATQRLANFLXHSSNNFGPILPPTRVGSNTY

SEQ ID NO: 156 - protein, 38 aa, synthetic construct
  REGION 1..38: note = Description of Artificial Sequence: Synthetic polypeptide
  MOD_RES 1: note = Lys, wherein the side chain of Lys is connected to gamma-Glu-C18diacid
  SITE 3..8: note = Disulphide bridge between Cys residues
  MOD_RES 15: note = 2,4-diaminobutanoic acid
  MOD_RES 18: note = 2-amino-2-methylpropanoic acid
XKCNTATCATQRLAXFLXHSSNNFGPILPPTEVGSNTY

SEQ ID NO: 157 - protein, 37 aa, synthetic construct; identical to SEQ ID NO: 89
  REGION 1..37: note = Description of Artificial Sequence: Synthetic polypeptide
  MOD_RES 1: note = Lys, wherein the side chain of Lys is connected to C18diacid
  SITE 2..7: note = Disulphide bridge between Cys residues
  MOD_RES 21: note = 2-amino-2-methylpropanoic acid
  MOD_RES 31: note = 2-amino-2-methylpropanoic acid

SEQ ID NO: 158 - protein, 37 aa, synthetic construct; identical to SEQ ID NO: 89
  REGION 1..37: note = Description of Artificial Sequence: Synthetic polypeptide
  MOD_RES 1: note = Lys, wherein the side chain of Lys is connected to C18diacid
  SITE 2..7: note = Disulphide bridge between Cys residues
  MOD_RES 21: note = 2-amino-2-methylpropanoic acid
  MOD_RES 31: note = 2-amino-2-methylpropanoic acid

SEQ ID NO: 159 - protein, 41 aa, synthetic construct; identical to SEQ ID NO: 2
  REGION 1..41: note = Description of Artificial Sequence: Synthetic polypeptide
  VARIANT 1: note = Lys (linker-lipid), or absent
  VARIANT 2: note = Gly or absent
  VARIANT 3: note = Gly or absent
  VARIANT 4: note = Gly, Lys (linker-lipid), or absent
  VARIANT 5: note = Lys, Lys (linker-lipid), or absent
  MOD_RES 8: note = Thr, Ile, Ala
  MOD_RES 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid
  SITE 1..41: note = Variant residues given in the sequence have no preference with respect to those in the annotations for variant positions

SEQ ID NO: 160 - protein, 37 aa, synthetic construct
  REGION 1..37: note = Description of Artificial Sequence: Synthetic polypeptide
  VARIANT 1: note = Lys or absent
  SITE 2..7: note = Disulphide bridge between Cys residues
  VARIANT 4: note = Thr, Ile, or Ala
  VARIANT 14: note = Asn, Glu, or His
  VARIANT 15: note = Phe or Trp
  VARIANT 17: note = Val, Arg, Ser, Glu
  VARIANT 20: note = Ser, Pro, or Ile
  VARIANT 21: note = Asn, His, Ala, Glu, Gly, Lys, Pro, Arg, or Ser
  VARIANT 22: note = Asn or His
  VARIANT 24: note = Gly or Pro
  VARIANT 25: note = Pro or Ala
  VARIANT 26: note = Ile or Arg
  VARIANT 28: note = Pro or Ser
  VARIANT 31: note = Asn, His, Pro, Arg, or Glu
  VARIANT 33: note = Gly or Pro
  VARIANT 34: note = Ser, His, or Pro
  VARIANT 35: note = Asn, Glu, Arg, Pro
  VARIANT 37: note = Tyr or Pro
XCNXATCATQRLAXXLXHSXXXFXXXLXPTXVXXXTX

SEQ ID NO: 161 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  MOD_RES 24: note = alpha methylSer
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 162 - protein, 40 aa, synthetic construct
  REGION 1..40: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 17: note = Asn, His, Glu, 2,4-diaminobutanoic acid (Dab), or an alpha methyl amino acid
  VARIANT 18: note = Phe or Trp
  VARIANT 19: note = Leu or D-Leu (dL)
  VARIANT 20: note = Val, Ser, Glu, Arg, (2S,4R)-4-hydroxypyrrolidine-2-carboxylic acid (Hyp), Dab or an alpha methyl amino acid
  VARIANT 23: note = Ser, Ile, Pro or an alpha methyl amino acid
  MOD_RES 24: note = Dab
  VARIANT 25: note = Asn, His, Hyp, Dab or an alpha methyl amino acid
  VARIANT 26: note = Phe, Hyp or an alpha methyl amino acid
  VARIANT 27: note = Gly, Pro, Hyp or an alpha methyl amino acid
  VARIANT 28: note = Pro, Ala, Hyp or an alpha methyl amino acid
  VARIANT 29: note = Ile, D-Ile (dI), Arg, Hyp or an alpha methyl amino acid
  VARIANT 30: note = Leu, dL, Hyp or an alpha methyl amino acid
  VARIANT 31: note = Pro, D-Pro (dP), Ser, Hyp or an alpha methyl amino acid
  VARIANT 32: note = Pro, Hyp or an alpha methyl amino acid
  VARIANT 34: note = Asn, Glu, His, Arg, Pro, Dab or an alpha methyl amino acid
  VARIANT 35: note = Val, Hyp, Dab or an alpha methyl amino acid
  VARIANT 36: note = Gly, Pro, Hyp or an alpha methyl amino acid
  VARIANT 37: note = Ser, Pro, His, Hyp or an alpha methyl amino acid
  VARIANT 39: note = Thr, Hyp or an alpha methyl amino acid
  VARIANT 40: note = Tyr, Pro, Hyp or an alpha methyl amino acid
XXXXCNIATCATQRLAXXXXHSXXXXXXXXXXTXXXXRXX

SEQ ID NO: 163 - protein, 40 aa, synthetic construct
  REGION 1..40: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 7: note = Thr, Ile or Ala
  VARIANT 17: note = Asn, His, Glu, 2,4-diaminobutanoic acid (Dab), or an alpha methyl amino acid
  VARIANT 18: note = Phe or Trp
  VARIANT 19: note = Leu or D-Leu (dL)
  VARIANT 20: note = Val, Ser, Glu, Arg, (2S,4R)-4-hydroxypyrrolidine-2-carboxylic acid (Hyp), Dab or an alpha methyl amino acid
  VARIANT 23: note = Ser, Ile, Pro or an alpha methyl amino acid
  MOD_RES 24: note = Aib
  VARIANT 25: note = Asn, His, Hyp, Dab or an alpha methyl amino acid
  VARIANT 26: note = Phe, Hyp or an alpha methyl amino acid
  VARIANT 27: note = Gly, Pro, Hyp or an alpha methyl amino acid
  VARIANT 28: note = Pro, Ala, Hyp or an alpha methyl amino acid
  VARIANT 29: note = Ile, D-Ile (dI), Arg, Hyp or an alpha methyl amino acid
  VARIANT 30: note = Leu, dL, Hyp or an alpha methyl amino acid
  VARIANT 31: note = Pro, D-Pro (dP), Ser, Hyp or an alpha methyl amino acid
  VARIANT 32: note = Pro, Hyp or an alpha methyl amino acid
  VARIANT 35: note = Val, Hyp, Dab or an alpha methyl amino acid
  VARIANT 36: note = Gly, Pro, Hyp or an alpha methyl amino acid
  VARIANT 37: note = Ser, Pro, His, Hyp or an alpha methyl amino acid
  VARIANT 38: note = Asn, Pro, Arg, Glu, Dab, Hyp or an alpha methyl amino acid
  VARIANT 39: note = Thr, Hyp or an alpha methyl amino acid
  VARIANT 40: note = Tyr, Pro, Hyp or an alpha methyl amino acid
XXXXCNXATCATQRLAXXXXHSXXXXXXXXXXTRXXXXXX

SEQ ID NO: 164 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  MOD_RES 18: note = 2,4-diaminobutanoic acid (Dab)
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 165 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  MOD_RES 20: note = dL
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid
XXXXXCNXATCATQRLAXXLXHSXXXXXXXXXXTXXXXXXX

SEQ ID NO: 166 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  MOD_RES 21: note = an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 167 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  MOD_RES 24: note = an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 168 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  MOD_RES 25: note = an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 169 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  MOD_RES 25: note = 2,4-diaminobutanoic acid (Dab)
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 170 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  MOD_RES 26: note = an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 171 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  MOD_RES 27: note = an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 172 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  MOD_RES 28: note = an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 173 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  MOD_RES 28: note = Hyp
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 174 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  MOD_RES 29: note = an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 175 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  MOD_RES 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 176 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  MOD_RES 30: note = dI
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid
XXXXXCNXATCATQRLAXXXXHSXXXXXXIXXXTXXXXXXX

SEQ ID NO: 177 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 178 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  MOD_RES 31: note = dL
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid
XXXXXCNXATCATQRLAXXXXHSXXXXXXXLXXTXXXXXXX

SEQ ID NO: 179 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  MOD_RES 32: note = an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 180 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  MOD_RES 32: note = dP
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid
XXXXXCNXATCATQRLAXXXXHSXXXXXXXXPXTXXXXXXX

SEQ ID NO: 181 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  MOD_RES 33: note = an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 182 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 183 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = 2,4-diaminobutanoic acid (Dab)
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 184 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  MOD_RES 36: note = an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 185 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  MOD_RES 37: note = an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 186 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  MOD_RES 38: note = an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 187 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = an alpha methyl amino acid
  VARIANT 40: note = Thr, Hyp or an alpha methyl amino acid
  VARIANT 41: note = Tyr, Pro, Hyp or an alpha methyl amino acid

SEQ ID NO: 188 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = 2,4-diaminobutanoic acid (Dab)
  VARIANT 40: note = Thr, Hyp or an alpha methyl amino acid
  VARIANT 41: note = Tyr, Pro, Hyp or an alpha methyl amino acid

SEQ ID NO: 189 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = an alpha methyl amino acid
  VARIANT 41: note = Tyr, Pro, Hyp or an alpha methyl amino acid

SEQ ID NO: 190 - protein, 41 aa, synthetic construct; identical to SEQ ID NO: 2
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 8: note = Thr, Ile or Ala
  VARIANT 18: note = Asn, His, Glu, 2,4-diaminobutanoic acid (Dab), or an alpha methyl amino acid
  VARIANT 19: note = Phe or Trp
  VARIANT 20: note = Leu or D-Leu (dL)
  VARIANT 21: note = Val, Ser, Glu, Arg, (2S,4R)-4-hydroxypyrrolidine-2-carboxylic acid (Hyp), 2,4-diaminobutanoic acid (Dab) or an alpha methyl amino acid
  VARIANT 24: note = Ser, Ile, Pro or an alpha methyl amino acid
  VARIANT 25: note = Asn, 2,4-diaminobutanoic acid (Dab), His, Pro, Ser, Arg, Lys, Gly or Glu, Ala, Hyp or an alpha methyl amino acid
  VARIANT 26: note = Asn, His, Hyp, 2,4-diaminobutanoic acid (Dab) or an alpha methyl amino acid
  VARIANT 27: note = Phe, Hyp or an alpha methyl amino acid
  VARIANT 28: note = Gly, Pro, Hyp or an alpha methyl amino acid
  VARIANT 29: note = Pro, Ala, Hyp or an alpha methyl amino acid
  VARIANT 30: note = Ile, D-Ile (dI), Arg, Hyp or an alpha methyl amino acid
  VARIANT 31: note = Leu, dL, Hyp or an alpha methyl amino acid
  VARIANT 32: note = Pro, D-Pro (dP), Ser, Hyp or an alpha methyl amino acid
  VARIANT 33: note = Pro, Hyp or an alpha methyl amino acid
  VARIANT 35: note = Asn, Glu, His, Arg, Pro, 2,4-diaminobutanoic acid (Dab) or an alpha methyl amino acid
  VARIANT 36: note = Val, Hyp, 2,4-diaminobutanoic acid (Dab) or an alpha methyl amino acid
  VARIANT 37: note = Gly, Pro, Hyp or an alpha methyl amino acid
  VARIANT 38: note = Ser, Pro, His, Hyp or an alpha methyl amino acid
  VARIANT 39: note = Asn, Pro, Arg, Glu, 2,4-diaminobutanoic acid (Dab), Hyp or an alpha methyl amino acid
  VARIANT 40: note = Thr, Hyp or an alpha methyl amino acid
  VARIANT 41: note = an alpha methyl amino acid

SEQ ID NO: 191 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Lys(albumin binding moiety) or absent
  VARIANT 5: note = Lys, Lys(albumin binding moiety) or absent
  VARIANT 18: note = Asn, Glu or 2,4-diaminobutanoic acid (Dab)
  VARIANT 21: note = Val, Arg or Aib
  VARIANT 27: note = Phe or alpha-MePhe
  VARIANT 35: note = Asn, Glu or Arg
  VARIANT 39: note = Asn or Arg
XXXXXCNTATCATQRLAXFLXHSSNNXGPILPPTXVGSXTY

SEQ ID NO: 192 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety) or absent
  VARIANT 5: note = Lys, Lys(albumin binding moiety) or absent
  VARIANT 18: note = Asn or His
  VARIANT 21: note = Val, Arg or Aib
  VARIANT 25: note = Asn, 2,4-diaminobutanoic acid (Dab) or Aib
  VARIANT 27: note = Phe or alpha-MePhe
  VARIANT 30: note = Ile or Aib
  VARIANT 31: note = Leu or Aib
  VARIANT 32: note = Pro or Aib
  VARIANT 33: note = Pro or Aib
  VARIANT 35: note = Asn, Glu or Aib
  VARIANT 36: note = Val or Aib
  VARIANT 37: note = Gly or Aib
  VARIANT 38: note = Ser or Aib
  VARIANT 39: note = Asn or Aib
  VARIANT 40: note = Thr or Aib
  VARIANT 41: note = Tyr, Pro or Aib
XXXXXCNTATCATQRLAXFLXHSSXNXGPXXXXTXXXXXXX

SEQ ID NO: 193 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Gly, Lys(albumin binding moiety), or absent
  VARIANT 5: note = Lys, Lys(albumin binding moiety) or absent
  VARIANT 8: note = Thr, Ile or Ala
  VARIANT 18: note = Asn, Glu or His
  VARIANT 20: note = Leu or D-Leu (dL)
  VARIANT 21: note = Val, Ser, Glu, Arg or Aib
  VARIANT 25: note = Asn or Aib
  VARIANT 28: note = Gly, Hyp or Pro
  VARIANT 29: note = Pro or Ala
  VARIANT 30: note = Ile, dI, or Aib
  VARIANT 31: note = Leu, dL, or Aib
  VARIANT 32: note = Pro, dP, Ser, or Aib
  VARIANT 35: note = Asn, Glu, His, Arg, Pro, 2,4-diaminobutanoic acid (Dab) or an alpha methyl amino acid
  VARIANT 37: note = Gly, Pro or Aib
  VARIANT 38: note = Ser or Aib
  VARIANT 39: note = Asn, Pro, Arg, Glu, 2,4-diaminobutanoic acid (Dab), or Aib
  VARIANT 40: note = Thr, or Aib
  VARIANT 41: note = Tyr, Pro or Aib
XXXXXCNXATCATQRLAXFXXHSSXNFXXXXXPTXVXXXXX

SEQ ID NO: 194 - protein, 41 aa, synthetic construct
  REGION 1..41: note = Description of Artificial Sequence: Synthetic polypeptide
  VARIANT 1: note = Lys(albumin binding moiety) or absent
  VARIANT 2: note = Gly or absent
  VARIANT 3: note = Gly or absent
  VARIANT 4: note = Lys(albumin binding moiety) or absent
  VARIANT 5: note = Lys, Lys(albumin binding moiety) or absent
  VARIANT 18: note = Asn, His, Glu, 2,4-diaminobutanoic acid (Dab), or an alpha methyl amino acid
  VARIANT 21: note = Val or Aib
  VARIANT 25: note = Asn, His, Pro, Ser, Arg, 2,4-diaminobutanoic acid (Dab), Lys or Gly
  VARIANT 26: note = Asn or His
  VARIANT 30: note = Ile or Arg
  VARIANT 31: note = Leu or dL
  VARIANT 35: note = Asn, Glu, His, Arg or Pro
  VARIANT 38: note = Ser, Pro or His
  VARIANT 39: note = Asn, Glu or Arg
XXXXXCNTATCATQRLAXFLXHSSXXFGPXXPPTXVGXXTY

SEQ ID NO: 195 - protein, 41 aa, synthetic construct; identical to SEQ ID NO: 2
  REGION 1..41: note = Description of Artificial Sequence: Synthetic polypeptide
  VARIANT 1: note = Lys (albumin binding moiety), or absent
  VARIANT 2: note = Gly or absent
  VARIANT 3: note = Gly or absent
  VARIANT 4: note = Gly, Lys (albumin binding moiety), or absent
  VARIANT 5: note = Lys, Lys (albumin binding moiety), or absent
  VARIANT 8: note = Thr, Ile, Ala
  VARIANT 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid
  SITE 1..41: note = At least one of positions 14, 17 or 20-37 comprises Dab, Hyp or an alpha methyl amino acid

SEQ ID NO: 196 - protein, 41 aa, synthetic construct; identical to SEQ ID NO: 2
  REGION 1..41: note = Description of Artificial Sequence: Synthetic polypeptide
  VARIANT 1: note = Lys (albumin binding moiety)
  VARIANT 2: note = Gly or absent
  VARIANT 3: note = Gly or absent
  VARIANT 4: note = Gly, Lys (albumin binding moiety), or absent
  VARIANT 5: note = Lys, Lys (albumin binding moiety), or absent
  VARIANT 8: note = Thr, Ile, Ala
  VARIANT 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid

SEQ ID NO: 197 - protein, 41 aa, synthetic construct; identical to SEQ ID NO: 2
  REGION 1..41: note = Description of Artificial Sequence: Synthetic polypeptide
  VARIANT 1: note = Lys (albumin binding moiety), or absent
  VARIANT 2: note = Gly or absent
  VARIANT 3: note = Gly or absent
  VARIANT 4: note = Lys (albumin binding moiety), or absent
  VARIANT 5: note = Lys, Lys (albumin binding moiety), or absent
  VARIANT 8: note = Thr, Ile, Ala
  VARIANT 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid

SEQ ID NO: 198 - protein, 41 aa, synthetic construct; identical to SEQ ID NO: 2
  REGION 1..41: note = Description of Artificial Sequence: Synthetic polypeptide
  VARIANT 1: note = Lys (albumin binding moiety), or absent
  VARIANT 2: note = Gly or absent
  VARIANT 3: note = Gly or absent
  VARIANT 4: note = Gly, Lys (albumin binding moiety), or absent
  VARIANT 5: note = Lys (albumin binding moiety)
  VARIANT 8: note = Thr, Ile, Ala
  VARIANT 18: note = Asn, His, Glu, 2,4-diaminobutanoic acid (Dab), alpha methyl amino acid
  VARIANT 19: note = Phe or Trp
  VARIANT 20: note = Leu or D-Leu
  VARIANT 21: note = Val, Ser, Glu, Arg, (2S,4R)-4-hydroxypyrrolidine -2-carboxylic acid (Hyp), Dab, alpha methyl amino acid
  VARIANT 24: note = Ser, Ile, Pro, alpha methyl amino acid
  VARIANT 25: note = Asn, Dab, His, Pro, Ser, Arg, Lys, Gly, Glu, Ala, Hyp , alpha methyl amino acid
  VARIANT 26: note = Asn, His, Hyp, Dab, alpha methyl amino acid
  VARIANT 27: note = Phe, Hyp, alpha methyl amino acid
  VARIANT 28: note = Gly, Pro, Hyp, alpha methyl amino acid
  VARIANT 29: note = Pro, Ala, Hyp, alpha methyl amino acid
  VARIANT 30: note = Ile, D-Ile, Arg, Hyp, alpha methyl amino acid
  VARIANT 31: note = Leu, D-Leu, Hyp, alpha methyl amino acid
  VARIANT 32: note = Pro, D-Pro, Ser, Hyp, alpha methyl amino acid
  VARIANT 33: note = Pro, Hyp, alpha methyl amino acid
  VARIANT 35: note = Asn, Glu, His, Arg, Pro, Dab, alpha methyl amino acid
  VARIANT 36: note = Val, Hyp, Dab, alpha methyl amino acid
  VARIANT 37: note = Gly, Pro, Hyp, alpha methyl amino acid
  VARIANT 38: note = Ser, Pro, His, Hyp, alpha methyl amino acid
  VARIANT 39: note = Asn, Pro, Arg, Glu, Dab, Hyp, alpha methyl amino acid
  VARIANT 40: note = Thr, Hyp, alpha methyl amino acid
  VARIANT 41: note = Tyr, Pro, Hyp, alpha methyl amino acid

SEQ ID NO: 199 - protein, 39 aa, synthetic construct
  REGION 1..39: note = Description of Artificial Sequence: Synthetic polypeptide
  SITE 4..9: note = Disulphide bridge between Cys residues
  VARIANT 19: note = 2-amino-2-methylpropanoic acid
GGKCNTATCATQRLANFLXHSSNNFGPILPPTNVGSNTY

SEQ ID NO: 200 - protein, 37 aa, synthetic construct
  REGION 1..37: note = Description of Artificial Sequence: Synthetic polypeptide
  SITE 2..7: note = Disulphide bridge between Cys residues
KCNAATCATQRLANWLVHSSPNFPAILSPTNVGSNTY

SEQ ID NO: 201 - protein, 37 aa, synthetic construct
  REGION 1..37: note = Description of Artificial Sequence: Synthetic polypeptide
  SITE 2..7: note = Disulphide bridge between Cys residues
KCNIATCATQRLANFLVHSIANFGPILPPTNVGSRTY

SEQ ID NO: 202 - protein, 37 aa, synthetic construct
  REGION 1..37: note = Description of Artificial Sequence: Synthetic polypeptide
  SITE 2..7: note = Disulphide bridge between Cys residues
  VARIANT 21: note = 2,4-diaminobutanoic acid (Dab)
KCNIATCATQRLANFLVHSSXNFGPILPPTNVGSRTY

SEQ ID NO: 203 - protein, 37 aa, synthetic construct
  REGION 1..37: note = Description of Artificial Sequence: Synthetic polypeptide
  SITE 2..7: note = Disulphide bridge between Cys residues
  VARIANT 14: note = 2,4-diaminobutanoic acid (Dab)
KCNTATCATQRLAXFLVHSSNNFGPILPPTNVGSNTY

SEQ ID NO: 204 - protein, 37 aa, synthetic construct
  REGION 1..37: note = Description of Artificial Sequence: Synthetic polypeptide
  SITE 2..7: note = Disulphide bridge between Cys residues
  VARIANT 14: note = 2,4-diaminobutanoic acid (Dab)
  VARIANT 17: note = 2-amino-2-methylpropanoic acid
KCNTATCATQRLAXFLXHSSNNFGPILPPTEVGSNTY

SEQ ID NO: 205 - protein, 37 aa, synthetic construct
  REGION 1..37: note = Description of Artificial Sequence: Synthetic polypeptide
  SITE 2..7: note = Disulphide bridge between Cys residues
  VARIANT 14: note = 2,4-diaminobutanoic acid (Dab)
  VARIANT 23: note = (S)-2-amino-2-methyl-3-phenylpropanoic acid
KCNTATCATQRLAXFLVHSSNNXGPILPPTEVGSNTY

SEQ ID NO: 206 - protein, 37 aa, synthetic construct
  REGION 1..37: note = Description of Artificial Sequence: Synthetic polypeptide
  SITE 2..7: note = Disulphide bridge between Cys residues
  VARIANT 14: note = 2,4-diaminobutanoic acid (Dab)
KCNTATCATQRLAXFLVHSSNNFGPILPPTEVGSNTY

SEQ ID NO: 207 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLAEFLXHSSNNFGPILPPTNVGSNTY

SEQ ID NO: 208 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLAEFLXHSSHNFGPILPPTNVGSNTY

SEQ ID NO: 209 - protein, 37 aa, synthetic construct
  REGION 1..37: note = Description of Artificial Sequence: Synthetic polypeptide
  SITE 2..7: note = Disulphide bridge between Cys residues
KCNTATCATQRLAEFLRHSSNNFGPILPPTNVGSNTY

SEQ ID NO: 210 - protein, 37 aa, synthetic construct
  REGION 1..37: note = Description of Artificial Sequence: Synthetic polypeptide
  SITE 2..7: note = Disulphide bridge between Cys residues
  VARIANT 23: note = (S)-2-amino-2-methyl-3-phenylpropanoic acid
KCNTATCATQRLAEFLRHSSNNXGPILPPTNVGSNTY

SEQ ID NO: 211 - protein, 37 aa, synthetic construct
  REGION 1..37: note = Description of Artificial Sequence: Synthetic polypeptide
  SITE 2..7: note = Disulphide bridge between Cys residues
  VARIANT 21: note = 2-amino-2-methylpropanoic acid
KCNTATCATQRLAEFLVHSSXNFGPILPPTNVGSNTY

SEQ ID NO: 212 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLAHFLXHSSNNFGPILPPTNVGSNTY

SEQ ID NO: 213 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
  VARIANT 21: note = 2-amino-2-methylpropanoic acid
KCNTATCATQRLAHFLXHSSXNFGPILPPTEVGSNTY

SEQ ID NO: 214 - protein, 37 aa, synthetic construct
  REGION 1..37: note = Description of Artificial Sequence: Synthetic polypeptide
  SITE 2..7: note = Disulphide bridge between Cys residues
  VARIANT 21: note = 2-amino-2-methylpropanoic acid
KCNTATCATQRLAHFLVHSSXNFGPILPPTNVGSNTY

SEQ ID NO: 215 - protein, 37 aa, synthetic construct
  REGION 1..37: note = Description of Artificial Sequence: Synthetic polypeptide
  SITE 2..7: note = Disulphide bridge between Cys residues
  VARIANT 21: note = 2-amino-2-methylpropanoic acid
KCNTATCATQRLAHFLVHSSXNFGPILPPTNVGSETY

SEQ ID NO: 216 - protein, 37 aa, synthetic construct
  REGION 1..37: note = Description of Artificial Sequence: Synthetic polypeptide
  SITE 2..7: note = Disulphide bridge between Cys residues
  VARIANT 16: note = D-Leu
  VARIANT 21: note = 2-amino-2-methylpropanoic acid
KCNTATCATQRLANFXVHSSXNFGPILPPTNVGSNTY

SEQ ID NO: 217 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLANFLXHSSNNFGPILPPTNVGSNTY

SEQ ID NO: 218 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLANFLXHSSNNFGPILPPTNVGSNTP

SEQ ID NO: 219 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
  VARIANT 21: note = 2-amino-2-methylpropanoic acid
KCNTATCATQRLANFLXHSSXNFGPILPPTNVGSNTY

SEQ ID NO: 220 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
  VARIANT 21: note = 2-amino-2-methylpropanoic acid
KCNTATCATQRLANFLXHSSXNFGPILPPTNVGSNTP

SEQ ID NO: 221 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLANFLXHSSGNFGPILPPTNVGSNTY

SEQ ID NO: 222 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLANFLXHSSHNFGPILPPTNVGSNTY

SEQ ID NO: 223 - protein, 37 aa, synthetic construct
  REGION 1..37: note = Description of Artificial Sequence: Synthetic polypeptide
  SITE 2..7: note = Disulphide bridge between Cys residues
  VARIANT 17: note = 2-amino-2-methylpropanoic acid
KCNTATCATQRLANFLXHSSKNFGPILPPTNVGSNTY

SEQ ID NO: 224 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSPNFGPILPPTNVGSNTY

SEQ ID NO: 225 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSPNFGPILPPTEVGSNTY

SEQ ID NO: 226 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSPNFGPILPPTNVGSETY

SEQ ID NO: 227 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSRNFGPILPPTNVGSNTY

SEQ ID NO: 228 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSSNFGPILPPTNVGSNTY

SEQ ID NO: 229 - protein, 37 aa, synthetic construct; identical to SEQ ID NO: 219
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 21: note = 2,4-diaminobutanoic acid (Dab)

SEQ ID NO: 230 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 21: note = 2,4-diaminobutanoic acid (Dab)
KCNTATCATQRLANFLXHSSXNFGPILPPTEVGSNTY

SEQ ID NO: 231 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNHFGPILPPTNVGSNTY

SEQ ID NO: 232 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNHFGPILPPTNVGSETY

SEQ ID NO: 233 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 23: note = (S)-2-amino-2-methyl-3-phenylpropanoic acid
KCNTATCATQRLANFLXHSSNNXGPILPPTNVGSNTY

SEQ ID NO: 234 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 26: note = 2-amino-2-methylpropanoic acid
KCNTATCATQRLANFLXHSSNNFGPXLPPTNVGSNTY

SEQ ID NO: 235 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPRLPPTNVGSNTY

SEQ ID NO: 236 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 27: note = 2-amino-2-methylpropanoic acid
KCNTATCATQRLANFLXHSSNNFGPIXPPTNVGSNTY

SEQ ID NO: 237 - protein, 37 aa, synthetic construct; identical to SEQ ID NO: 236
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 27: note = D-Leu

SEQ ID NO: 238 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 28: note = 2-amino-2-methylpropanoic acid
KCNTATCATQRLANFLXHSSNNFGPILXPTNVGSNTY

SEQ ID NO: 239 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 29: note = 2-amino-2-methylpropanoic acid
KCNTATCATQRLANFLXHSSNNFGPILPXTNVGSNTY

SEQ ID NO: 240 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 31: note = 2-amino-2-methylpropanoic acid
KCNTATCATQRLANFLXHSSNNFGPILPPTXVGSNTY

SEQ ID NO: 241 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTEVGSNTY

SEQ ID NO: 242 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTHVGSETY

SEQ ID NO: 243 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTPVGSNTY

SEQ ID NO: 244 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTRVGSNTY

SEQ ID NO: 245 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 32: note = 2-amino-2-methylpropanoic acid
KCNTATCATQRLANFLXHSSNNFGPILPPTNXGSNTY

SEQ ID NO: 246 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 33: note = 2-amino-2-methylpropanoic acid
KCNTATCATQRLANFLXHSSNNFGPILPPTNVXSNTY

SEQ ID NO: 247 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 34: note = 2-amino-2-methylpropanoic acid
KCNTATCATQRLANFLXHSSNNFGPILPPTNVGXNTY

SEQ ID NO: 248 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTNVGHNTY

SEQ ID NO: 249 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTNVGPNTY

SEQ ID NO: 250 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
  MOD_RES 35: note = 2-amino-2-methylpropanoic acid
KCNTATCATQRLANFLXHSSNNFGPILPPTNVGSXTY

SEQ ID NO: 251 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTNVGSETY

SEQ ID NO: 252 - protein, 37 aa, synthetic construct
  REGION 1..37: note = Description of Artificial Sequence: Synthetic polypeptide
  SITE 2..7: note = Disulphide bridge between Cys residues
  MOD_RES 17: note = 2-amino-2-methylpropanoic acid
KCNTATCATQRLANFLXHSSNNFGPILPPTNVGSRTY

SEQ ID NO: 253 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLEHSSXNFGPILPPTNVGSNTY

SEQ ID NO: 254 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLRHSSXNFGPILPPTNVGSNTY

SEQ ID NO: 255 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 31: note = 2-amino-2-methylpropanoic acid
KCNTATCATQRLANFLRHSSXNFGPILPPTXVGSNTY

SEQ ID NO: 256 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLRHSSXNFGPILPPTEVGSNTY

SEQ ID NO: 257 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLRHSSXNFGPILPPTRVGSNTY

SEQ ID NO: 258 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 35: note = 2-amino-2-methylpropanoic acid
KCNTATCATQRLANFLRHSSXNFGPILPPTNVGSXTY

SEQ ID NO: 259 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLRHSSNNXGPILPPTNVGSNTY

SEQ ID NO: 260 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLRHSSNNXGPILPPTEVGSNTY

SEQ ID NO: 261 - protein, 37 aa, synthetic construct
  REGION 1..37: note = Description of Artificial Sequence: Synthetic polypeptide
  SITE 2..7: note = Disulphide bridge between Cys residues
  MOD_RES 26: note = 2-amino-2-methylpropanoic acid
KCNTATCATQRLANFLRHSSNNFGPXLPPTNVGSNTY

SEQ ID NO: 262 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTNVGSNTY

SEQ ID NO: 263 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTHVGSNTY

SEQ ID NO: 264 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTPVGSNTY

SEQ ID NO: 265 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTRVGSNTY

SEQ ID NO: 266 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTNVPSNTY

SEQ ID NO: 267 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLSHSSXNFGPILPPTNVGSPTY

SEQ ID NO: 268 - protein, 37 aa, synthetic construct
  REGION 1..37: note = Description of Artificial Sequence: Synthetic polypeptide
  SITE 2..7: note = Disulphide bridge between Cys residues
  MOD_RES 20: note = (S)-2-amino-3-hydroxy-2-methylpropanoic acid
KCNTATCATQRLANFLVHSXNNFGPILPPTNVGSNTY

SEQ ID NO: 269 - protein, 37 aa, synthetic construct
  REGION 1..37: note = Description of Artificial Sequence: Synthetic polypeptide
  SITE 2..7: note = Disulphide bridge between Cys residues
KCNTATCATQRLANFLVHSPPNFPAILSPTNVGSNTY

SEQ ID NO: 270 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTNVGSNTY

SEQ ID NO: 271 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFPAILSPTNVGSNTY

SEQ ID NO: 272 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 31: note = 2,4-diaminobutanoic acid (Dab)
KCNTATCATQRLANFLVHSSXNFPAILSPTXVGSNTY

SEQ ID NO: 273 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 35: note = 2,4-diaminobutanoic acid (Dab)
KCNTATCATQRLANFLVHSSXNFPAILSPTNVGSXTY

SEQ ID NO: 274 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 26: note = D-Ile
KCNTATCATQRLANFLVHSSXNFGPXLPPTNVGSNTY

SEQ ID NO: 275 - protein, 37 aa, synthetic construct; identical to SEQ ID NO: 274
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 26: note = 2-amino-2-methylpropanoic acid

SEQ ID NO: 276 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 27: note = 2-amino-2-methylpropanoic acid
KCNTATCATQRLANFLVHSSXNFGPIXPPTNVGSNTY

SEQ ID NO: 277 - protein, 37 aa, synthetic construct; identical to SEQ ID NO: 276
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 27: note = D-Leu

SEQ ID NO: 278 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 28: note = 2-amino-2-methylpropanoic acid
KCNTATCATQRLANFLVHSSXNFGPILXPTNVGSNTY

SEQ ID NO: 279 - protein, 37 aa, synthetic construct; identical to SEQ ID NO: 278
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 28: note = D-Pro

SEQ ID NO: 280 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 31: note = 2-amino-2-methylpropanoic acid
KCNTATCATQRLANFLVHSSXNFGPILPPTXVGSNTY

SEQ ID NO: 281 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTEVGSNTY

SEQ ID NO: 282 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTHVGSNTY

SEQ ID NO: 283 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTRVGSNTY

SEQ ID NO: 284 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 33: note = 2-amino-2-methylpropanoic acid
KCNTATCATQRLANFLVHSSXNFGPILPPTNVXSNTY

SEQ ID NO: 285 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 34: note = 2-amino-2-methylpropanoic acid
KCNTATCATQRLANFLVHSSXNFGPILPPTNVGXNTY

SEQ ID NO: 286 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 35: note = 2-amino-2-methylpropanoic acid
KCNTATCATQRLANFLVHSSXNFGPILPPTNVGSXTY

SEQ ID NO: 287 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTNVGSETY

SEQ ID NO: 288 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTNVGSRTY

SEQ ID NO: 289 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 36: note = 2-amino-2-methylpropanoic acid
KCNTATCATQRLANFLVHSSXNFGPILPPTNVGSNXY

SEQ ID NO: 290 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 37: note = 2-amino-2-methylpropanoic acid
KCNTATCATQRLANFLVHSSXNFGPILPPTNVGSNTX

SEQ ID NO: 291 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
KCNTATCATQRLANFLVHSSXNFGPILPPTNVGSNTP

SEQ ID NO: 292 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2-amino-2-methylpropanoic acid
  MOD_RES 24: note = (2S,4R)-4-hydroxypyrrolidine-2-carboxylic acid
KCNTATCATQRLANFLVHSSXNFXAILSPTNVGSNTY

SEQ ID NO: 293 - protein, 37 aa, synthetic construct; identical to SEQ ID NO: 271
  REGION 1..37: note = Description of Artificial Sequence: Synthetic polypeptide
  SITE 2..7: note = Disulphide bridge between Cys residues
  MOD_RES 21: note = 2,4-diaminobutanoic acid (Dab)

SEQ ID NO: 294 - protein, 37 aa, synthetic construct
  REGION 1..37: note = Description of Artificial Sequence: Synthetic polypeptide
  SITE 2..7: note = Disulphide bridge between Cys residues
  MOD_RES 21: note = 2,4-diaminobutanoic acid (Dab)
  MOD_RES 25: note = 2-amino-2-methylpropanoic acid
KCNTATCATQRLANFLVHSSXNFGXILPPTNVGSNTY

SEQ ID NO: 295 - protein, 37 aa, synthetic construct; identical to SEQ ID NO: 281
  REGION 1..37: note = Description of Artificial Sequence: Synthetic polypeptide
  SITE 2..7: note = Disulphide bridge between Cys residues
  MOD_RES 21: note = 2,4-diaminobutanoic acid (Dab)

SEQ ID NO: 296 - protein, 37 aa, synthetic construct
  REGION 1..37: note = Description of Artificial Sequence: Synthetic polypeptide
  SITE 2..7: note = Disulphide bridge between Cys residues
KCNTATCATQRLANFLVHSSPNFPAILSPTNVGSNTY

SEQ ID NO: 297 - protein, 37 aa, synthetic construct
  REGION 1..37: note = Description of Artificial Sequence: Synthetic polypeptide
  SITE 2..7: note = Disulphide bridge between Cys residues
  MOD_RES 22: note = 2-amino-2-methylpropanoic acid
KCNTATCATQRLANFLVHSSNXFGPILPPTNVGSNTY

SEQ ID NO: 298 - protein, 37 aa, synthetic construct
  REGION 1..37: note = Description of Artificial Sequence: Synthetic polypeptide
  SITE 2..7: note = Disulphide bridge between Cys residues
KCNTATCATQRLANFLVHSSNHFGPILPPTNVGSETY

SEQ ID NO: 299 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLVHSSNNXGPILPPTNVGSNTY

SEQ ID NO: 300 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLVHSSNNXGPILPPTEVGSNTY

SEQ ID NO: 301 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLVHSSNNXGPILPPTRVGSNTY

SEQ ID NO: 302 - protein, 37 aa, synthetic construct
  REGION 1..37: note = Description of Artificial Sequence: Synthetic polypeptide
  SITE 2..7: note = Disulphide bridge between Cys residues
  MOD_RES 23: note = (S)-2-amino-2-methyl-3-phenylpropanoic acid
KCNTATCATQRLANFLVHSSNNXGPILPPTNVGSRTY

SEQ ID NO: 303 - protein, 37 aa, synthetic construct
  REGION 1..37: note = Description of Artificial Sequence: Synthetic polypeptide
  SITE 2..7: note = Disulphide bridge between Cys residues
  MOD_RES 24: note = 2-amino-2-methylpropanoic acid
KCNTATCATQRLANFLVHSSNNFXPILPPTNVGSNTY

SEQ ID NO: 304 - protein, 37 aa, synthetic construct
  REGION 1..37: note = Description of Artificial Sequence: Synthetic polypeptide
  SITE 2..7: note = Disulphide bridge between Cys residues
  MOD_RES 26: note = 2-amino-2-methylpropanoic acid
KCNTATCATQRLANFLVHSSNNFGPXLPPTNVGSNTY

SEQ ID NO: 305 - protein, 37 aa, synthetic construct
  REGION 1..37: note = Description of Artificial Sequence: Synthetic polypeptide
  SITE 2..7: note = Disulphide bridge between Cys residues
  MOD_RES 27: note = 2-amino-2-methylpropanoic acid
KCNTATCATQRLANFLVHSSNNFGPIXPPTNVGSNTY

SEQ ID NO: 306 - protein, 37 aa, synthetic construct; identical to SEQ ID NO: 305
  REGION 1..37: note = Description of Artificial Sequence: Synthetic polypeptide
  SITE 2..7: note = Disulphide bridge between Cys residues
  MOD_RES 27: note = D-Leu

SEQ ID NO: 307 - protein, 37 aa, synthetic construct
  REGION 1..37: note = Description of Artificial Sequence: Synthetic polypeptide
  SITE 2..7: note = Disulphide bridge between Cys residues
  MOD_RES 28: note = D-Pro
KCNTATCATQRLANFLVHSSNNFGPILXPTNVGSNTY

SEQ ID NO: 308 - protein, 37 aa, synthetic construct
  REGION 1..37: note = Description of Artificial Sequence: Synthetic polypeptide
  SITE 2..7: note = Disulphide bridge between Cys residues
KCNTATCATQRLANFLVHSSNNFGPILPPTNVGSRTY

SEQ ID NO: 309 - protein, 36 aa, synthetic construct
  REGION 1..36: note = Description of Artificial Sequence: Synthetic polypeptide
  SITE 1..6: note = Disulphide bridge between Cys residues
  MOD_RES 20: note = 2,4-diaminobutanoic acid (Dab)
CNIATCATQRLANFLVHSSXNFGPILPPTNVGSRTY

SEQ ID NO: 310 - protein, 36 aa, synthetic construct
  REGION 1..36: note = Description of Artificial Sequence: Synthetic polypeptide
  SITE 1..6: note = Disulphide bridge between Cys residues
  MOD_RES 22: note = (S)-2-amino-2-methyl-3-phenylpropanoic acid
CNTATCATQRLAEFLRHSSNNXGPILPPTNVGSNTY

SEQ ID NO: 311 - protein, 36 aa, synthetic construct
  REGION 1..36: note = Description of Artificial Sequence: Synthetic polypeptide
  SITE 1..6: note = Disulphide bridge between Cys residues
  MOD_RES 20: note = 2-amino-2-methylpropanoic acid
CNTATCATQRLANFLVHSSXNFGPILPPTRVGSNTY